(19) United States
(12) Patent Application Publication
(10) Pub. No.: US 2005/0032713 A1
(43) Pub. Date: Feb. 10, 2005

(54) Platelet-activating factor antagonists as analgesic, anti-inflammatory, uterine contraction inhibiting, and anti-tumor agents

(76) Inventors: Richard J. Wurtman, Boston, MA (US); Lisa A. Teather, St. Catharine's (CA)

Correspondence Address: EITAN, PEARL, LATZER & COHEN ZEDEK LLP, 10 ROCKEFELLER PLAZA, SUITE 1001, NEW YORK, NY, 10020, US

(21) Appl. No.: 10/890,387

(22) Filed: Jul. 14, 2004

Related U.S. Application Data
(63) Continuation-in-part of application No. 10/397,228, filed on Mar. 27, 2003.
(60) Provisional application No. 60/367,488, filed on Mar. 27, 2002.

(51) Int. Cl. A61K 31/704

(52) U.S. Cl. 514/26; 514/33

(57) ABSTRACT

Antagonists to platelet-activating factor provide analgesic effects as well as limit the release of inflammatory mediators. Use of these antagonists in the form of pharmaceutical compositions or nutritionals is beneficial (1) in the treatment of acute and/or chronic pain; (2) in the inhibition of inappropriate or excessive contraction of the uterus; (3) in the treatment of septic shock; and (4) in the inhibition of angiogenesis and/or tumor cell proliferation.

### CROSS REFERENCE TO RELATED APPLICATIONS

This application is a Continuation-in-Part of U.S. application Ser. No. 10/397,228, filed Mar. 27, 2003, which is incorporated herein by reference.

### STATEMENT REGARDING FEDERALLY SPONSORED RESEARCH OR DEVELOPMENT

The invention described herein was supported in part by grants from The National Institutes of Mental Health (Grant No. 5-RO1 MH28783-24) and The Center for Brain Sciences and Metabolism Charitable Trust.

### FIELD OF THE INVENTION

This invention relates generally to beneficial effects obtained via administration of antagonists to platelet-activating factor. In particular, this invention relates to treatment of acute or chronic pain, inhibition of inappropriate or excessive contraction of the uterus, treatment of septic shock, and inhibition of angiogenesis.

### BACKGROUND OF THE INVENTION

Platelet Activating Factor, or PAF (1-0-alkyl-2-acetyl-sn-glycero-3-phosphocholine) is a family of structurally related and biologically potent phospholipid mediators. PAF is a membrane-derived mediator that has biological effects on a variety of cells and tissues. A variety of stimuli, including those producing inflammation, promote the synthesis and release of PAF from various cell types. PAF is synthesized in and released by various cells in the PNS and CNS.

PAF exerts cellular actions through high affinity intracellular membrane-binding sites, as well as through low-affinity cell surface receptors. The binding of PAF to cell surface receptors results in the activation of diverse intracellular signal transduction pathways that ultimately activate transcription factors and induce gene expression For example, calcium, cyclic AMP (cAMP), inositol 1,4,5-triphosphate (IP₃), and diacylglycerol (DAG) can function as second messengers for signaling by the plasma membrane PAF receptor. Moreover, PAF also acts as an intracellular mediator, binding to intracellular sites, which then elicit gene expression in neuronal and glial cell lines.

While early animal studies relating to PAF antagonists were encouraging, more recent studies have been disappointing. Thus, The Pharmacologic Basis of Therapeutics states “[ . . . ] it appears as though currently available PAF antagonists are of little benefit in human disease.” (Goodman & Gilman, The Pharmacologic Basis of Therapeutics 10^{th} edition, 2001, edited by J. Hardman and L. Limbird, p. 682.) Novel anti-inflammatory drugs are urgently needed to treat a wide variety of inflammation-mediated disorders.

### SUMMARY OF THE INVENTION

In another embodiment, the present invention provides a method of treating pain, comprising blocking or inhibiting a cell surface PAF receptor, wherein said cell surface PAF receptor is in the brain.

In another embodiment, the present invention provides a method of treating pain, comprising blocking or inhibiting an intracellular PAF receptor, wherein said intracellular PAF receptor is in the spinal cord.

In another embodiment, the present invention provides a method of treating inflammation, comprising blocking or inhibiting a cell surface PAF receptor, wherein said cell surface PAF receptor is in the brain.

In another embodiment, the present invention provides a method of treating inflammation, comprising blocking or inhibiting an intracellular PAF receptor, wherein said intracellular PAF receptor is in the spinal cord.

In another embodiment, the present invention provides a method of inhibiting a contraction of a uterus in a subject, comprising blocking or inhibiting a cell surface PAF receptor, wherein said cell surface PAF receptor is in the brain.

In another embodiment, the present invention provides a method of inhibiting a contraction of a uterus in a subject, comprising blocking or inhibiting an intracellular PAF receptor, wherein said intracellular PAF receptor is in the spinal cord.

In another embodiment, the present invention provides a method of inhibiting a proliferation of a tumor cell in a subject, comprising blocking or inhibiting a cell surface PAF receptor, wherein said cell surface PAF receptor is in the brain.

In another embodiment, the present invention provides a method of inhibiting a proliferation of a tumor cell in a subject, comprising blocking or inhibiting an intracellular PAF receptor, wherein said intracellular PAF receptor is in the spinal cord.

In another embodiment, the present invention provides a method of inhibiting angiogenesis in a subject, comprising blocking or inhibiting a cell surface PAF receptor, wherein said cell surface PAF receptor is in the brain.

In another embodiment, the present invention provides a method of inhibiting angiogenesis in a subject, comprising blocking or inhibiting an intracellular PAF receptor, wherein said intracellular PAF receptor is in the spinal cord.

In another embodiment, the present invention provides a method of inhibiting neural damage in a subject, comprising blocking or inhibiting a cell surface PAF receptor, wherein said cell surface PAF receptor is in the brain.

In another embodiment, the present invention provides a method of inhibiting neural damage in a subject, comprising blocking or inhibiting an intracellular PAF receptor, wherein said intracellular PAF receptor is in the spinal cord.

The present invention relates to methods of controlling or alleviating pain by controlling activation of astrocytes and/or other cell types and thus preventing these cells from releasing harmful substances that kill or overexcite surrounding neurons.

The present invention also relates to the use of PAF antagonists that act preferentially at the cell surface site in diseases involving excitotoxicity, such as ischemia and stroke.

The present invention also relates to the use of PAF antagonists that act preferentially at the intracellular binding sites in inflammatory/immune-based disorders, such as sepsis, Alzheimer's, ALS, multiple sclerosis.

The present invention also relates to the combined use of PAF antagonists having different selectivity in those diseases or disorders where PAF is having pathological effect at both surface and intracellular sites.

In one aspect of the present invention, a method is provided for the use of drugs or nutritional supplements to diminish pain or inflammation comprising blocking one or more receptors for platelet-activating factor.

In another aspect of the present invention, a method is provided for the use of drugs or nutritional supplements to diminish pain or inflammation comprising blocking one or more cell surface receptors for platelet-activating factor and/or by blocking one or more intracellular receptor binding sites for platelet-activating factor.

In another aspect of the present invention, a method is provided for the use of nutritional supplements related to Gingko biloba and its constituents to diminish pain or inflammation comprising blocking one or more receptors for platelet-activating factor.

In another aspect of the present invention, a method is provided for the use of synthetic drugs related to benzodiazapines to diminish pain or inflammation comprising blocking one or more receptors for platelet-activating factor.

In another aspect of the present invention, a method is provided for the use of synthetic drugs related to tetrahydrofurans to diminish pain or inflammation comprising blocking one or more receptors for platelet-activating factor.

In another aspect of the present invention, a method is provided for the use of BN 52021, BN 50730, WEB 286, CV 6209, CV 3988, trans-2,5-Bis(3,4,5-trimethoxypenyl)-1,3-dioxolane, 1-O-hexadecyl-2-O-acetyl-sn-glycero-3-phospho(N,N,N-trimethyl) hexanolamine, octylonium bromide, PCA-4248, and tetrahydrocannabinol-7-oic acid, either alone or in combination, to diminish pain or inflammation comprising blocking one or more receptors for platelet-activating factor.

In another aspect of the present invention, a method is provided for the use of compounds that inhibit prostaglandin synthesis by decreasing or abolishing platelet-activating factor actions to treat pain.

In another aspect of the present invention, a method is provided for the use of drugs or nutritional supplements to inhibit the inappropriate or excessive contraction of the uterus comprising blocking one or more receptors for platelet-activating factor.

In another aspect of the present invention, a method is provided for inhibiting pain and/or cramps associated with premenstrual syndrome (also known as late luteal phase dysphoric disorder, or premenstrual dysphoric disorder) comprising blocking one or more receptors for platelet-activating factor.

In another aspect of the present invention, a method is provided for inhibiting pain and/or cramps associated with normal menses comprising blocking one or more receptors for platelet-activating factor.

In another aspect of the present invention, a method is provided for inhibiting spontaneous abortion/miscarriage comprising blocking one or more receptors for platelet-activating factor.

In another aspect of the present invention, a method is provided for inhibiting pain, cramping, and/or discomfort associated with the perimenopausal period comprising blocking one or more receptors for platelet-activating factor.

In another aspect of the present invention, a method is provided for reducing pain associated with childbirth, including pain experienced during and post labor comprising blocking one or more receptors for platelet-activating factor.

In another aspect of the present invention, a method is provided for inhibiting Braxton Hicks contractions comprising blocking one or more receptors for platelet-activating factor.

In another aspect of the present invention, a method is provided for inhibiting the initiation and/or the severity of septic shock comprising one or more blocking receptors for platelet-activating factor.

In another aspect of the present invention, a method is provided for inhibiting the proliferation of tumor cells comprising blocking receptors for platelet-activating factor.

In another aspect of the present invention, a method is provided for inhibiting neo-angiogenesis comprising blocking one or more receptors for platelet-activating factor.

### BRIEF DESCRIPTION OF THE DRAWINGS

FIG. 1 illustrates time-dependent PGE2 release induced by mc-PAF from astrocyte-enriched cortical cell cultures. Cells were incubated with 1 μM mc-PAF or vehicle (0.01% methanol) at 37° C. for various times. Media was collected and assayed for PGE2. Each point represents the mean+/−Standard Error of the Mean (SEM) of at least 3 independent experiments, carried out in duplicate or triplicate. “*” indicates statistically significant (p<0.05) differences relative to control.

FIGS. 2A and B illustrate PGE2 release from astrocyte-enriched cortical cell cultures exposed to (FIG. 2A) mc-PAF, lyso-PAF, PAF-16, or PAF-18 and (FIG. 2B) mc-PAF, lyso-PC or PC. Cells were incubated in the respective treatments at 37° C. for 30 min, at which time the media was collected and assayed for PGE2 (as described in materials and methods). Each point represents the mean+/−SEM of at least 3 independent experiments, carried out in duplicate or triplicate. The mean+/−SEM for control cultures was 35.6+/−7.9, and significant differences are indicated in results section.

FIGS. 3A, B, and C illustrate concentration-dependent PGE2 release from media of astrocyte-enriched cortical cell cultures exposed to (FIG. 3A) AA (0.01-10 μM) and (FIG. 3B) AA (0.01 μM) with or without mc-PAF (0.01-1 μM) and (FIG. 3C) AA (10 μM) with or without mc-PAF (0.01-1 μM). Cells were incubated in various concentrations of AA (with or without mc-PAF) or vehicle (0.01% ethanol, 0.01% methanol or both) at 37° C. for 30 min, at which time the media was collected and assayed for PGE2 (as described in materials and methods). Each point represents the mean+/−SEM of at least 3 independent experiments, carried out in duplicate or triplicate. * indicates statistically significant (p<0.05) differences relative to control and ** relative to AA alone.

FIGS. 4A, B, and C illustrate the PAF antagonist, BN 50730 attenuates the (FIG. 4A) mc-PAF-, (FIG. 4B) lyso-PAF- and (FIG. 4C) AA- induced PGE2 release in astrocytes in concentration-dependent manners. Cells were incubated at 37° C. for 30 minute (min) with various concentrations of BN 50730 before addition of mc-PAF (1 μM). After 30 min, media was collected and assayed for PGE2 (as described in materials and methods). Each point represents the mean+/−SEM of at least 3 independent experiments, carried out in duplicate or triplicate. * indicates statistically significant (p<0.05) differences relative to control and ** relative to mc-PAF, lyso-PAF or AA alone.

FIGS. 5A and B illustrate the PAF antagonists, (FIG. 5A) BN 52021 (1-50 μM) and (FIG. 5B) CV 6209 (1-50 μM) do not attenuate the mc-PAF-induced PGE2 release in astrocytes in concentration-dependent manners. Cells were incubated at 37° C. for 30 min in the respective antagonists before addition of mc-PAF (1 μM. After 30 min, media was collected and assayed for PGE2 (as described in materials and methods). Each point represents the mean+/−SEM of at least 3 independent experiments, carried out in duplicate or triplicate. * indicates statistically significant (p<0.05) differences relative to control.

FIGS. 6A and B illustrate formalin-evoked nociceptive responses in rats that receive systemic BN 52021 (10, 1 or 0.1 mg/kg) or control injections. Total paw elevation times in (FIG. 6A) the early phase (0-10 min after injection) and (FIG. 6B) the late phase (10-60 min after injection) of formalin-induced nociception. Data are expressed as means=/−SEM. * p<0.05; Fisher's PLSD test vs control. HBC=45% hydroxypropyl-B-cyclodextrin (in distilled water).

FIGS. 7A and B illustrate formalin-evoked nociceptive responses in rats that received systemic BN 50730 (10, 1 or 0.1 mg/kg) or control injections. Total paw elevation times in (FIG. 7A) the early phase (0-10 min after injection) and (FIG. 7B) the late phase (10-60 mins after injection) of formalin-induced nociception. Data are expressed as means=/−SEM. * p<0.05; Fisher's PLSD test vs control. HBC=45% hydroxypropyl-B-cyclodextrin (in distilled water).

FIG. 8 illustrates the effect of PGE2 release from primary cortical astrocytes exposed to the non-hydrolyzable analog of PAF, methylcarbamyl-PAF (mc-PAF). Cells were incubated at 37° C. with various mc-PAF concentrations for 30 min, at which time the media was collected and assayed for PGE2. Each point represents the mean+/−S.E.M. of at least three independent experiments, carried out in triplicate. The mean+/−S.E.M. for control cultures was 0.8+/−0.011.

FIGS. 9A and 9B illustrate that preferential COX-1-selective inhibitors have minimal influence on the mc-PAF-induced PGE2 release from astrocytes. Cells were incubated at 37° C. with various concentrations of (A) piroxicam or (B) SC-560 for 30 min prior to addition of mc-PAF (0.1 uM) for 30 min, at which time the media was collected and assayed for PGE2. Each point represents the mean+/−S.E.M. of at least three independent experiments, carried out in triplicate. *, Statistically significant (P<0.05) difference relative to control and **, relative to mc-PAF.

FIGS. 10A and 10B illustrate that inhibition of COX-2 attenuates mc-PAF-induced PGE2 release from astrocytes. Cells were incubated at 37° C. with various concentrations of (A) the noselective COX inhibitor indomethacin or (B) the COX-2 selective inhibitor NS-398 for 30 minutes prior to addition of mc-PAF (0.1 uM) for 30 min, at which time the media was collected and assayed for PGE2. Each point represents the mean+/−S.E.M. of at least three independent experiments, carried out in triplicate. “*” denotes a statistically significant (P<0.05) difference relative to control and ** denotes a statistically significant difference relative to mc-PAF.

FIGS. 11A and 11B. Intracellular, but not plasma membrane, PAF binding sites mediate nociception in the spinal cord. Formalin-evoked nociceptive responses in rats that received intra-thecal injections of A) BN 52021 (10, 1 or 0.1 μg) or B) BN 50730 (10, 1 or 0.1 μg) 20 minutes prior to formalin. Data are expressed as means=/−SEMs.

FIGS. 12A and 12B. Plasma membrane, but not intracellular, PAF binding sites mediate nociception in the brain. Formalin-evoked nociceptive responses in rats that received intra-ventricular injections of A) BN 52021 (10, 1 or 0.1 μg) or B) BN 50730 (10, 1 or 0.1 μg) 20 minutes prior to formalin. Data are expressed as means=/−SEMs.

### DETAILED DESCRIPTION OF THE INVENTION

Prostaglandins (PGs) have important functions in brain cells, and mediate a variety of neuropathologic phenomena, including such inflammation-associated disorders as Alzheimer's disease (AD) and amyotrophic lateral sclerosis (ALS). When cells and tissue are exposed to various stimuli, arachidonic acid (AA) is liberated from membrane phospholipids and is converted to prostanoids, including PGs, by the action of cyclooxygenase (COX) enzymes. Two related but unique isoforms of COX, cyclooxygenase-1 (COX-1) and cyclooxygenase-2 (COX-2) catalyze identical reactions, a cyclooxygenation to form PGG2, and a peroxidation which reduces PGG2 to PGH2, the precursor of all other PGs, including PGE2. COX-1 is constitutively expressed by most cells and is considered to be involved in maintaining cell homeostasis; in contrast the mitogen-inducible COX-2 is implicated in inflammatory and immune responses.

Astrocytes have an important role in CNS inflammation/immune responses. Following CNS injury or an immune/inflammatory challenge, astrocytes undergo a phenotypic alteration—a response known as activation. The activated astrocytes then release cytokines and other pro-inflammatory mediators, including PGs. These released substances communicate with (and ultimately affect the function of) such neighboring cells as neurons and microvascular cells. Astrocytes are a major source of PGs in the CNS; in culture these cells synthesize up to 20 times more PGs than do neurons. PGE2 is the major AA metabolite involved in modulation of immunoinflammatory responses.

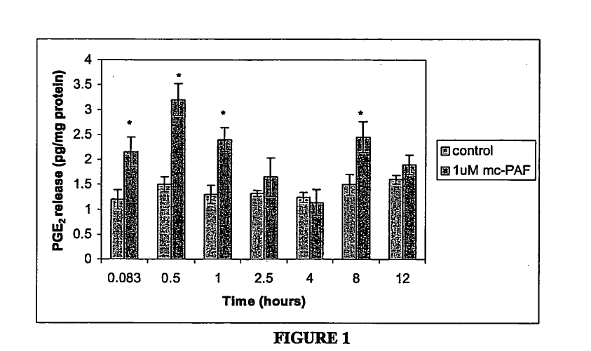

The acute or immediate phase of inflammation is the earliest response to tissue injury, as well as to immunological or pro-inflammatory challenges. COX-1 is often responsible for the immediate increases in PGs produced by various types of inflammatory stimuli, and COX-2 for the increased levels characteristic of the delayed phase of inflammation. However, the degree to which each COX isoform contributes to particular acute inflammatory responses depends upon such factors as the nature of the inflammatory stimulus and the cell type involved. PAF, acting at micosomal binding sites, increases the release of PGE2 from cortical astrocytes. This effect is observed within minutes of PAF stimulation, and PGE2 accumulation peaks at 30 minutes, suggesting that PAF induces an acute inflammatory reaction in astrocytes. As FIG. 1 shows, PAF increases PGE2 release at 8 hours. Therefore, PAF may also have a role in delayed inflammatory reactions as well.

The experimental results set forth herein describe a role for endogenous PAF in nociceptive transmission, especially for persistent pain. The findings also indicate that both intracellular and cell surface PAF binding sites are involved in nociceptive modulation in rats, and that PAF antagonists are useful for treating patients having acute or chronic pain. As described herein, the nociceptive responses to subcutaneous formalin injection are significantly reduced in rats receiving PAF antagonists that act on intracellular or cell surface PAF binding sites. In one embodiment of the present invention, treatment comprises administration of at least two PAF antagonists, each having selectivity for a different receptor. One receptor may be sufficient for treatment, depending on the type of pain being treated. For some types of pain, however, the use of two antagonists may be required.

The effect of PAF and the PAF antagonists is assessed on the release of prostaglandin E2 (PGE2) from astrocytes. Also disclosed herein is the participation of the two COX isozymes in PAF-induced PGE2 mobilization, using COX inhibitors with varying degrees of selectivity for COX-1 and COX-2. It has been suggested that activated astrocytes are responsible for the majority of the increased arachidonic acid and eicosanoid levels. The PAF-induced PGE2 release initiates an inflammatory cascade in astrocytes that can be detrimental to cell function and/or kill surrounding neurons. By preventing such actions by endogenous PAF, PAF antagonists may, in one embodiment, alleviate pain and provide neuroprotection in various disorders of the nervous system that are caused by or aggravated by inflammatory mediator production.

In one embodiment, the present invention provides a method of treating pain, comprising blocking or inhibiting a cell surface PAF receptor, wherein said cell surface PAF receptor is in the brain. The present invention demonstrates that BN 52021, a PAF antagonist that inhibits cell-surface PAF receptors, was more effective than BN 50730, a PAF antagonist that inhibits intracellular PAF receptors, when administered to the brain. By contrast, BN 50730 was more effective than BN 52021 when administered to the spinal cord (Example 4). Thus, a method is provided of increasing the efficacy of treatment of a PAF-mediated or PAF-related disease or disorder by administering a PAF antagonist that inhibits cell-surface PAF receptors to the brain. In another embodiment, the PAF antagonist may be administered to another tissue in which inhibition of cell-surface PAF receptors decreases a PAF-mediated disease or disorder. In another embodiment, the PAF antagonist may possess an ability to localize to brain or another tissue in which inhibition of cell-surface PAF receptors decreases a PAF-mediated disease or disorder. In this case, in one embodiment, the PAF antagonist need not be administered to the target tissue. Each of these methods represents a separate embodiment of the present invention.

In one embodiment, the cell surface PAF receptor may be located in the cerebellum or the hippocampus. In another embodiment, the cell surface PAF antagonist may be located in another region of the brain. In another embodiment, the cell surface PAF antagonist may be located in another tissue in which inhibition of cell-surface PAF receptors decreases a PAF-mediated disease or disorder. Each of these methods represents a separate embodiment of the present invention.

In another embodiment, the PAF antagonist or a pharmaceutical composition or nutritional supplement comprising same may be administered to or targeted to the lateral ventricle or the hippocampus. In another embodiment, the PAF antagonist or a pharmaceutical composition or nutritional supplement comprising same may be administered to or targeted to another tissue in which inhibition of cell-surface PAF receptors decreases a PAF-mediated disease or disorder. In another embodiment, the PAF antagonist or a pharmaceutical composition or nutritional supplement comprising same may be administered to or targeted to another location at or near the target tissue. In another embodiment, the PAF antagonist or a pharmaceutical composition or nutritional supplement comprising same may be administered to or targeted to another location from which it will travel or diffuse to the target tissue. Each of these methods represents a separate embodiment of the present invention.

In one embodiment, the PAF antagonist that inhibits cell-surface PAF receptors may be BN 52021. In another embodiment, the PAF antagonist that inhibits cell-surface PAF receptors may be CV-3988, CV-3938, CV-6209, TCV-309, E5880, SRI 63-441, ginkgolide A, ginkgolide B, ginkgolide C, ginkgolide T, ginkgolide M, or a derivative thereof, either alone or in combination. In another embodiment, the PAF antagonist that inhibits cell-surface PAF receptors may be a nutritional supplement comprising Gingko biloba, or derivatives or constitutents thereof. PAF antagonists structurally or functionally related to BN 52021 are expected, in one embodiment, to also be effecting in inhibiting PAF-mediated or PAF-related diseases and/or disorders when administered or targeted to the brain or a similar tissue. Each of these methods represents a separate embodiment of the present invention

In another embodiment, the present invention provides a method of treating pain, comprising blocking or inhibiting an intracellular PAF receptor, wherein said intracellular PAF receptor is in the spinal cord. In another embodiment, the intracellular PAF antagonist may be administered to another tissue in which inhibition of intracellular PAF receptors decreases a PAF-mediated disease or disorder. In another embodiment, the intracellular PAF antagonist may possess an ability to localize to brain or another tissue in which inhibition of intracellular PAF receptors decreases a PAF-mediated disease or disorder. In this case, in one embodiment, the intracellular PAF antagonist need not be administered to the target tissue. Each of these methods represents a separate embodiment of the present invention.

In one embodiment, the intracellular PAF receptor may be located in the spinal cord. In another embodiment, the intracellular PAF antagonist may be located in another tissue in which inhibition of intracellular PAF receptors decreases a PAF-mediated disease or disorder. Each of these methods represents a separate embodiment of the present invention.

In another embodiment, the PAF antagonist or a pharmaceutical composition or nutritional supplement comprising same may be administered to or targeted to the spinal cord. In another embodiment, the PAF antagonist or a pharmaceutical composition or nutritional supplement comprising same may be administered to or targeted to another tissue in which inhibition of intracellular PAF receptors decreases a PAF-mediated disease or disorder. In another embodiment, the PAF antagonist or a pharmaceutical composition or nutritional supplement comprising same may be administered to or targeted to another location at or near the target tissue. In another embodiment, the PAF antagonist or a pharmaceutical composition or nutritional supplement comprising same may be administered to or targeted to another location from which it will travel or diffuse to the target tissue. Each of these methods represents a separate embodiment of the present invention.

In one embodiment, the PAF antagonist that inhibits intracellular PAF receptors may be BN 50730. In another embodiment, the PAF antagonist that inhibits intracellular PAF receptors may be WEB-2086, WEB-2170, Y-24180, BN 50727, BN 50730, BN 50739, and E 6123, SM-12502, YM264, ABT-299, SR 27417, SRI 63-073, ONO-6240, RO-19 3704, UK-74,505, BB-882, or a derivative thereof, either alone or in combination. PAF antagonists structurally or functionally related to BN 52021 are expected, in one embodiment, to also be effecting in inhibiting PAF-mediated or PAF-related diseases and/or disorders when administered or targeted to the spinal cord or a similar tissue. Each of these methods represents a separate embodiment of the present invention.

In another embodiment, the present invention provides a method of treating inflammation, comprising blocking or inhibiting a cell surface PAF receptor, wherein said cell surface PAF receptor is in the brain.

In another embodiment, the present invention provides a method of treating inflammation, comprising blocking or inhibiting an intracellular PAF receptor, wherein said intracellular PAF receptor is in the spinal cord.

In another embodiment, the present invention provides a method of inhibiting a contraction of a uterus in a subject, comprising blocking or inhibiting a cell surface PAF receptor, wherein said cell surface PAF receptor is in the brain.

In another embodiment, the present invention provides a method of inhibiting a contraction of a uterus in a subject, comprising blocking or inhibiting an intracellular PAF receptor, wherein said intracellular PAF receptor is in the spinal cord.

In another embodiment, the present invention provides a method of inhibiting a proliferation of a tumor cell in a subject, comprising blocking or inhibiting a cell surface PAF receptor, wherein said cell surface PAF receptor is in the brain.

In another embodiment, the present invention provides a method of inhibiting a proliferation of a tumor cell in a subject, comprising blocking or inhibiting an intracellular PAF receptor, wherein said intracellular PAF receptor is in the spinal cord.

In another embodiment, the present invention provides a method of inhibiting angiogenesis in a subject, comprising blocking or inhibiting a cell surface PAF receptor, wherein said cell surface PAF receptor is in the brain.

In another embodiment, the present invention provides a method of inhibiting angiogenesis in a subject, comprising blocking or inhibiting an intracellular PAF receptor, wherein said intracellular PAF receptor is in the spinal cord.

In another embodiment, the present invention provides a method of inhibiting neural damage in a subject, comprising blocking or inhibiting a cell surface PAF receptor, wherein said cell surface PAF receptor is in the brain.

In another embodiment, the present invention provides a method of inhibiting neural damage in a subject, comprising blocking or inhibiting an intracellular PAF receptor, wherein said intracellular PAF receptor is in the spinal cord.

The present invention relates to methods of controlling or alleviating pain by controlling activation of astrocytes and/or other cell types and thus preventing these cells from releasing harmful substances that kill or overexcite surrounding neurons.

The present invention also relates to the use of PAF antagonists that act preferentially at the cell surface site in diseases involving excitotoxicity; such as ischemia and stroke.

The present invention also relates to the use of PAF antagonists that act preferentially at the intracellular binding sites in inflammatory/immune-based disorders, such as sepsis, alzheimer's, ALS, multiple sclerosis.

The present invention also relates to the combined use of PAF antagonists having different selectivity in those diseases or disorders where PAF is having pathological effect at both surface and intracellular sites.

In one aspect of the present invention, a method is provided for the use of drugs or nutritionals to diminish pain or inflammation comprising blocking one or more receptors for platelet-activating factor.

In another aspect of the present invention, a method is provided for the use of drugs or nutritionals to diminish pain or inflammation comprising blocking one or more cell surface receptors for platelet-activating factor and/or by blocking one or more intracellular receptor binding sites for platelet-activating factor.

In another aspect of the present invention, a method is provided for the use of nutritionals related to Gingko biloba and its constituents to diminish pain or inflammation comprising blocking one or more receptors for platelet-activating factor.

In another aspect of the present invention, a method is provided for the use of synthetic drugs related to benzodiazapines to diminish pain or inflammation comprising blocking one or more receptors for platelet-activating factor.

In another aspect of the present invention, a method is provided for the use of synthetic drugs related to tetrahydrofurans to diminish pain or inflammation comprising blocking one or more receptors for platelet-activating factor.

In another aspect of the present invention, a method is provided for the use of BN 52021, BN 50730, WEB 286, CV 6209, CV 3988, trans-2,5-Bis(3,4,5-trimethoxypenyl)-1,3-dioxolane, 1-O-hexadecyl-2-O-acetyl-sn-glycero-3-phospho(N,N,N-trimethyl) hexanolamine, octylonium bromide, PCA-4248, and tetrahydrocannabinol-7-oic acid, either alone or in combination, to diminish pain or inflammation comprising blocking one or more receptors for platelet-activating factor.

In another aspect of the present invention, a method is provided for the use of compounds that inhibit prostaglandin synthesis by decreasing or abolishing platelet-activating factor actions to treat pain.

In another aspect of the present invention, a method is provided for the use of drugs or nutritionals to inhibit the inappropriate or excessive contraction of the uterus comprising blocking one or more receptors for platelet-activating factor.

In another aspect of the present invention, a method is provided for inhibiting pain and/or cramps associated with premenstrual syndrome (also known as late luteal phase dysphoric disorder, or premenstrual dysphoric disorder) comprising blocking one or more receptors for platelet-activating factor.

In another aspect of the present invention, a method is provided for inhibiting pain and/or cramps associated with normal menses comprising blocking one or more receptors for platelet-activating factor.

In another aspect of the present invention, a method is provided for inhibiting spontaneous abortion/miscarriage comprising blocking one or more receptors for platelet-activating factor.

In another aspect of the present invention, a method is provided for inhibiting pain, cramping, and/or discomfort associated with the perimenopausal period comprising blocking one or more receptors for platelet-activating factor.

In another aspect of the present invention, a method is provided for reducing pain associated with childbirth, including pain experienced during and post labor comprising blocking one or more receptors for platelet-activating factor.

In another aspect of the present invention, a method is provided for inhibiting Braxton Hicks contractions comprising blocking one or more receptors for platelet-activating factor.

In another aspect of the present invention, a method is provided for inhibiting the initiation and/or the severity of septic shock comprising one or more blocking receptors for platelet-activating factor.

In another aspect of the present invention, a method is provided for inhibiting the proliferation of tumor cells comprising blocking receptors for platelet-activating factor.

In another aspect of the present invention, a method is provided for inhibiting neo-angiogenesis comprising blocking one or more receptors for platelet-activating factor.

The above-recited methods are accomplished by the administration of a therapeutically effective amount of one or more antagonists to platelet activating factor that either block one or more receptors for platelet-activating factors or block one or more binding sites for platelet-activating factors. These antagonists can be pharmaceuticals or nutraceuticals. Each of the above-recited diseases and disorders is mediated by, involves, or is caused by PAF.

For each of the above-recited methods of the present invention, the therapeutically effective amount of one or more PAF antagonists may be administered in conjunction with a therapeutically effective amount of one or more anti-inflammatory compounds and/or a therapeutically effective amount of one or more immunomodulatory agents.

In certain embodiments of the method of the present invention, the anti-inflammatory compound or immunomodulatory drug comprises interferon; interferon derivatives comprising betaseron, .beta.-interferon; prostane derivatives comprising iloprost, cicaprost; glucocorticoids comprising cortisol, prednisolone, methyl-prednisolone, dexamethasone; immunsuppressives comprising cyclosporine A, FK-506, methoxsalene, thalidomide, sulfasalazine, azathioprine, methotrexate; lipoxygenase inhibitors comprising zileutone, MK-886, WY-50295, SC-45662, SC-41661A, BI-L-357; leukotriene antagonists; peptide derivatives comprising ACTH and analogs thereof; soluble TNF-receptors; TNF-antibodies; soluble receptors of interleukines, other cytokines, T-cell-proteins; antibodies against receptors of interleukines, other cytokines, T-cell-proteins; and calcipotriols and analogues thereof taken either alone or in combination.

In one aspect of the invention, the therapeutically effective amount of the one or more antagonists to platelet activating factor administered is that amount sufficient to reduce or inhibit, inter alia, the pain associated with one or more of the following diseases: ischemia, stroke, sepsis, amyotrophic lateral sclerosis (ALS), epilepsy, extension of strokes after initial tissue damage, Alzheimer's disease, Parkinson's disease, Huntington's disease, functional brain damage secondary to primary and secondary brain tumors, Pick's disease, diffuse Lewy body disease, progressive supranuclear palsy, cerebellar degeneration, Shy-drager syndrome, amyotrophic lateral sclerosis, spinal muscular atrophy, multiple sclerosis, local brain damage secondary to meningitis or brain abscess, viral meningitis, viral encephalitis, HIV neurological disease, and/or local brain damage secondary to trauma.

In another aspect of the invention, the therapeutically effective amount of the one or more antagonists to platelet activating factor administered is that amount sufficient to inhibit the inappropriate or excessive contraction of the uterus, inhibit the pain and/or cramps associated with premenstrual syndrome (also known as late luteal phase dysphoric disorder, or premenstrual dysphoric disorder), inhibit the pain and/or cramps associated with normal menses, inhibit spontaneous abortion/miscarriage, inhibit the pain, cramping, and/or discomfort associated with the perimenopausal period, reduce the pain associated with childbirth, including pain experienced during and post labor, inhibit Braxton Hicks contractions, inhibit the initiation and/or the severity of septic shock, inhibit the proliferation of tumor cells, and/or inhibit neo-angiogenesis.

In one embodiment, the reduction or inhibition of pain and/or symptoms associated with one or more of each of the above-recited indications is on the order of about 10-20% reduction or inhibition. In another embodiment, the reduction or inhibition of pain is on the order of 30-40%. In another embodiment, the reduction or inhibition of pain is on the order of 50-60%. In another embodiment, the reduction or inhibition of the pain associated with each of the recited indications is on the order of 75-100%. It is intended herein that the ranges recited also include all those specific percentage amounts between the recited range. For example, the range of about 75 to 100% also encompasses 76 to 99%, 77 to 98%, etc, without actually reciting each specific range therewith.

In yet another aspect, the present invention is directed to a method of relieving or ameliorating the pain or symptoms associated with any one or more of the above-identified diseases or indications in a mammal suffering from any one or more of the above-identified diseases or indications which comprises administering to the mammal in need thereof a therapeutically effective pain or symptom-reducing amount of a pharmaceutical composition comprising one or more antagonists to platelet activating factor, either alone or in combination with one or more anti-inflammatory compounds or immunomodulatory agents; and a pharmaceutically acceptable carrier or excipient.

In one aspect of the invention, the one or more one or more antagonists to platelet activating factor of the present invention are administered orally, systemically, via an implant, intravenously, topically, or intrathecally.

In certain embodiments of the methods of the present invention, the subject or mammal is a human.

In other embodiments of the methods of the present invention, the subject or mammal is a veterinary and/or a domesticated mammal.

There has been thus outlined, rather broadly, the important features of the invention in order that a detailed description thereof that follows can be better understood, and in order that the present contribution can be better appreciated. There are additional features of the invention that will be described hereinafter.

### PAF Antagonists

In one embodiment, any PAF antagonist may be utilized in the present invention. PAF antagonists include natural products (naturally occurring PAF-antagonists including chemical derivatives of terpenes, lignans and gliotoxins), synthetic structural analogs of PAF, synthetic PAF antagonist compounds that have thiazolidine/thiazole and pyridine moieties, synthetic PAF antagonist compounds that have methylimidazopyridine moieties, and synthetic small molecule PAF antagonists, and any other compounds that possesses the activity of PAF antagonist.

### Natural Products

An example of naturally occurring PAF antagonists are the ginkgolides A, B, and C, T, and M. These compounds are terpenoids derived from the leaves of Ginkgo biloba, and are competitive PAF antagonists. The Ginkgo biloba tree of Gingkoaceae is of the gymnosperm order Ginkoales. Of these, ginkolide B is the strongest PAF antagonist, and is commercially available under the name BN52021 (IHB, Research Labs, France, among other commercial companies).

Plants of the Zingiberaceae species, including but not limited to, Alphinia galanga, Boesenbergia pandurata, Curcuma aeruginosa, C. domestica, C. ochorrhiza, C. xanthorriza, Aingiber officinale, and Z. zerumbet have effects similar to the Gingko Biloba extracts. Other sources of PAF antagonists include the cinnamomum species such as Cinnamomum altissimum, C. aureofulvum, and C. pubescens, as well as Ardisia elliptica, Goniothalamus malayanus, Kopsia flavida, Momordica charantia and Piper aduncem. Lastly, the bark extract of Drymis winteri of the Winteraceae family contains a sesquiterpene with anti-inflammatory and anti-allergic properties.

Another ligand with PAF antagonist activity is kadsurenone (from Piper Futokadsurae, South China). It is orally active and is reported to have potent antagonist activity in a number of systems. A structural analogue, L-65 2731 (Merck Sharp and Dome) has considerably enhanced potency.

Fermentation of some fungi and microorganisms have produced antagonists which are structurally related to the gliotoxins. The most potent antagonist are FR 900 452 (S phacofaciens) and FR-49175 (F. testikowski).

PAF antagonists of the tetrahydrofuran class include L659,989 (trans-2-(3-methoxy-5-methylsulfonyl-4-propoxyphenyl)-5(3,4,5-trimethoxyphenyl) tetrahydrofuran); MK 287 (L-680,573); and magnone A ((2S, 3R, 4R)-tetrahydro-2-(3,4-dimethoxyphenyl)-4-(3,4-dimethoxybenzoyl)-3-(hydroxymethyl)furan) and magnone B ((2S, 3R, 4R)-tetrahydro-2-(3,4,5-trimethoxyphenyl)-4-(3,4-dimethoxybenzoyl)-3-(hydroxymethyl)furan) from the flower buds of Magnolia fargesii.

PAF antagonists of the benzodiazepine class include WEB-2086, WEB-2170, Y-24180, BN 50727, BN 50730, BN 50739, and E 6123. The triazolobenzodiazepines, particularly Alprazolam and Triazolam potently inhibit PAF activity in vitro. Structural alteration of the triazolobenzodiazepines has resulted in production of numbers of potent antagonists of which WEB 2086 (Boehringer Ingelhelm) is the most widely studied.

### Synthetic Structural Analogs of PAF

Synthetic compounds with structures similar to PAF include CV-3988, CV-3938, CV-6209, TCV-309, E5880, and SRI 63-441. The most widely used and one of the first PAF antagonists developed is CV-3988 which incorporates an octadecyl carbamate in position 1, a methylether in position 2 and thiazolium ethyl phosphate in position 3. It is orally active in most systems tested and is relatively potent. At very high dose it may antagonise arachidonic acid and ADP activation of platelets.

### Other Synthetic Structures

Synthetic compounds useful as PAF antagonists having thiazolidine/thiazole and pyridine moieties include SM-12502, YM264, ABT-299, SR 27417. Cyclization of the PAF structure has resulted in another series SRI 63-073 (Sandoz). A heptamethylene thiazolium at C.sub.3 gave a potent antagonist termed ONO-6240. Other minor alterations to this basic structure have been performed by Hoffman La Roche and RO-19 3704 is the best of these antagonists. Synthetic compounds useful as PAF antagonists having methylimidazopyridine moiety include UK-74,505 and BB-882 (Lexipafant). WEB 2086 (Apafant) is derived from an anxioilytic triazolobenzodiazepine. WEB 2086 related compounds include Y-24180, BN 50727, BN 50730, BN 50739, and E 6123. Lastly, GM2 activator protein is a good candidate for the development of small molecule PAF antagonists. Each PAF antagonist, natural product with PAF antagonist activity, or structural analogue of PAF with PAF antagonist activity represents a separate embodiment of the present invention.

### PAF-Mediated Conditions or PAF-Related Conditions

In another aspect, the present invention provides pharmaceutical compositions useful for the treatment of PAF-mediated disorders comprising a therapeutically effective amount of a PAF antagonist compound in combination with a pharmaceutically acceptable carrier.

In another aspect, the present invention provides a method of inhibiting PAF activity by administering to a host mammal in need of such treatment an effective amount of a PAF-antagonist compound.

In yet another aspect of the present invention, there is provided a method of treating a PAF-mediated disorder or PAF-related disorder including ischemia and stroke, sepsis, inhibit the inappropriate or excessive contraction of the uterus, inhibit pain and/or cramps associated with premenstrual syndrome (also known as late luteal phase dysphoric disorder, or premenstrual dysphoric disorder), inhibit pain and/or cramps associated with normal menses, inhibit spontaneous abortion/miscarriage, inhibit pain, cramping, and/or discomfort associated with the perimenopausal period, reduce pain associated with childbirth, including pain experienced during and post labor, inhibit Braxton Hicks contractions, inhibit the initiation and/or the severity of septic shock, inhibit the proliferation of tumor cells, inhibit neo-angiogenesis by administering to a host mammal in need of such treatment a therapeutically effective amount of PAF antagonist compound.

Other important indications for a PAF antagonist include the following: epilepsy, extension of strokes after initial tissue damage, Alzheimer's disease, Parkinson's disease, Huntington's disease, functional brain damage secondary to primary and secondary brain tumors, Pick's disease, diffuse Lewy body disease, progressive supranuclear palsy, cerebellar degeneration, Shy-drager syndrome, amyotrophic lateral sclerosis, spinal muscular atrophy, multiple sclerosis, local brain damage secondary to meningitis or brain abscess, viral meningitis, viral encephalitis, HIV neurological disease, local brain damage secondary to trauma. Each PAF-mediated or PAF-related condition represents a separate embodiment of the present invention.

Yet other important indications for a PAF antagonist include the following: inflammatory processes of the tracheobronchial tree (acute and chronic bronchitis, bronchial asthma) or of the kidneys (glomerulonephritis), the joints (rheumatic complaints), anaphylactic conditions, allergies and inflammation in the mucous membranes (rhinitis, conjunctivitis) and the skin (e.g. psoriasis, atopic eczema, cold-induced urticaria, allergic dermatitis) and shock caused by sepsis, endotoxins, trauma or burns, lesions and inflammation in the gastric and intestinal linings, such as shock ulcers, ulcerative colitis, Crohn's disease, ischemic bowel necrosis, stress ulcers and peptic ulcers in general, but particularly ventricular and duodenal ulcers; obstructive lung diseases such as bronchial hyper-reactivity; inflammatory diseases of the pulmonary passages, such as chronic bronchitis; cardio/circulatory diseases such as polytrauma, anaphylaxis and arteriosclerosis; inflammatory intestinal diseases, EPH gestosis (edema-proteinuria hypertension); diseases of extracorporeal circulation, e.g. heart insufficiency, cardiac infarct, organ damage caused by high blood pressure, ischaemic diseases (i.e., cerebral, myocardial and renal ischemia), inflammatory and immunological diseases (i.e. rheumatoid arthritis); immune modulation in the transplanting of foreign tissues, e.g. the rejection of kidney, liver an other transplants; immune modulation in leukemia; propagation of metastasis, e.g. in bronchial neoplasia; diseases of the CNS, such as migraine, multiple sclerosis, endogenic depression and agarophobia (panic disorder). The PAF antagonist compounds of the present invention could also be effective as cyto- and organoprotective agents, e.g. for neuroprotection; to treat DIC (disseminated intravascular coagulation); to treat side effects of drug therapy, e.g. anaphylactoid circulatory reactions; to treat incidents caused by contrast media and other side effects in tumor therapy; to diminish incompatibilities in blood transfusions; to prevent fulminant liver failure (CCl₄ intoxication); to treat amanita phalloides intoxication (mushroom poisoning); to treat symptoms of parasitic diseases (e.g. worms); to treat autoimmune diseases (e.g. Werlhof s disease); to treat autoimmune hemolytic anemia, autoimmunologically induced glomerulonephritis, thyroids Hashimoto, primary myxoedema, pernicious anemia, autoimmune atrophic gastritis, Addison's disease, juvenile diabetes, Goodpasture syndrome, idiopathic leukopenia, primary biliary cirrhosis, active or chronically aggressive hepatitis (HBsAg-neg.), ulcerative colitis and systemic lupus erythematodes (SLE), idiopathic thrombocytopenic purpura (ITP); to treat diabetes, diabetic retinopathy, polytraumatic shock, haemorrhagic shock; to treat thrombocytopenia, endotoxin shock, adult respiratory distress syndrome; and to treat PAF-associated interaction with tissue hormones (autocoid hormones), lymphokines and other mediators; and any other condition in which PAF is implicated. Each of these indications represents a separate embodiment of the present invention.

The PAF antagonist compounds of the present invention may also be used in combinations for which PAF-antagonists are suitable, e.g. with .beta.-adrenergics, parasympatholytics, corticosteroids, antiallergic agents and secretolytics. When the PAF antagonist compounds of the present invention are combined with TNF (tumor necrosis factor), the TNF may, in one embodiment, be better tolerated (elimination of disturbing side effects). Thus, TNF may, in one embodiment, be used in higher dosages than when it is administered alone. The term “combination” here also includes the administration of the two active substances in separate preparations simultaneously or in sequence over a time period. When compounds are administered in combination with .beta.-adrenergics, a synergistic effect may be achieved. Each combination represents a separate embodiment of the present invention.

### Mode of Administration and Pharmaceutical Compositions

The compounds of the present invention include pharmaceutically acceptable salts that can be prepared by those of skill in the art. As used herein, “pharmaceutically acceptable salt” refers to, in one embodiment, those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M Berge, et al. describe pharmaceutically acceptable salts in detail in J. Pharmaceutical Sciences, 1977, 66: 1-19. The salts can be prepared in situ during the final isolation and purification of the compounds of the invention, or separately by reacting the free base function with a suitable organic acid. Representative acid addition salts include acetate, adipate, alginate, ascorbate, aspartate, benzene-sulfonate, benzoate, bisulfate, borate, butyrate, camphorate, camphersulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary as ammonium, and mine cations, including, but not limited to ammonium, tetramethylammonium, tetraethylammonium, methylamine, dimethylamine, trimethylamine, triethylamine, ethylamine, and the like.

The present invention also provides pharmaceutical compositions which comprise one or more of the PAF antagonist compounds described above formulated together with one or more non-toxic pharmaceutically acceptable carriers. The pharmaceutical compositions may be specially formulated for oral administration in solid or liquid form, for parenteral injection, or for rectal administration.

The pharmaceutical compositions of this invention can be administered to humans and other animals orally, rectally, parenterally, intracistemally, intravaginally, intraperitoneally, topically (as by powders, ointments, or drops), bucally, or as an oral or nasal spray. The term “parenteral” administration as used herein refers to modes of administration which include intravenous, intramuscular, intraperitoneal, intrathecally, intrasternal, subcutaneous and intraarticular injection and infusion.

Pharmaceutical compositions of this invention for parenteral injection comprise pharmaceutically acceptable sterile aqueous or nonaqueous solutions, dispersions, suspensions, or emulsions as well as sterile powders for reconstitution into sterile injectable solutions or dispersions just prior to use. Examples of suitable aqueous and nonaqueous carders, diluents, solvents, or vehicles include water, ethanol, polyols (such as glycerol, propylene glycol, polyethylene glycol, and the like), and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

These compositions may also contain adjuvants such as preservative, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents which delay absorption such as aluminum monostearate and gelatin.

In some cases, in order to prolong the effect of the drug, it is desirable to slow the absorption of the drug from subcutaneous or intramuscular injection. This may be accomplished by the use of a liquid suspension of crystalline or amorphous material with poor water solubility. The rate of absorption of the drag then depends upon its rate of dissolution which, in turn, may depend upon crystal size and crystalline form. Alternatively, delayed absorption of a parenterally administered drug form is accomplished by dissolving or suspending the drag in an oil vehicle.

Injectable depot forms are made by forming microencapsule matrices of the drag in biodegradable polymers such as polylactide-polyglycolide. Depending upon the ratio of drag to polymer and the nature of the particular polymer employed, the rate of drag release can be controlled. Examples of other biodegradable polymers include poly(orthoesters) and poly(anhydrides). Depot injectable formulations are also prepared by entrapping the drag in liposomes or microemulsions which are compatible with body tissues.

The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidone, sucrose, and acacia, (c) humectants such as glycerol, (d) disintegrating agents such as agar-agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

The active compounds can also be in micro-encapsulated form, if appropriate, with one or more of the above-mentioned excipients.

Liquid dosage forms for oral administration include pharmaceutically acceptable emulsions, solutions, suspensions, syrups, and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, and perfuming agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Compositions for rectal or vaginal administration are, in one embodiment, suppositories which can be prepared by mixing the compounds of this invention with suitable non-irritating excipients or carriers such as cocoa butter, polyethylene glycol, or a suppository wax which are solid at room temperature but liquid at body temperature and therefore melt in the rectum or vaginal cavity and release the active compound.

The PAF antagonist compounds of the present invention can also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The present compositions in liposome form can contain, in addition to a PAF antagonist compound of the present invention, stabilizers, preservatives, excipients, and the like. In one embodiment, the lipids may be the phospholipids and the phosphatidyl cholines (lecithins), both natural and synthetic.

Methods to form liposomes are known in the art. See, for example, Prescott, Ed., Methods in Cell Biology, Volume XIV, Academic Press, New York, N.Y. (1976), p. 33 et seq.

Dosage forms for topical administration of a PAF antagonist compound of this invention include powders, sprays, ointments, and inhalants. The active compound is mixed under sterile conditions with a pharmaceutically acceptable carrier and any needed preservatives, buffers, or propellants which may be required. Opthalmic formulations, eye ointments, powders and solutions are also contemplated as being within the scope of this invention.

Actual dosage levels of active ingredients in the pharmaceutical compositions of this invention may be varied so as to obtain an amount of the active compound(s) that is effective to achieve the desired therapeutic response for a particular patient, compositions, and mode of administration. The selected dosage level will depend as upon the activity of the particular PAF antagonist compound, the route of administration, the severity of the condition being treated, and the condition and prior medical history of the patient being treated. However, it is within the skill of the art to start doses of the PAF antagonist compound at levels lower than required to achieve the desired therapeutic effect and to gradually increase the dosage until the desired effect is achieved.

The pharmaceutical compositions of the present invention can be used in both veterinary medicine and human therapy. The magnitude of a prophylactic or therapeutic dose of the pharmaceutical composition of the invention in the acute or chronic management of pain associated with above-mentioned diseases or indications will vary with the severity of the condition to be treated and the route of administration. The dose, and perhaps the dose frequency, will also vary according to the age, body weight, and response of the individual patient. In one embodiment, the total daily dose range of the PAF antagonist compound of this invention is between about 0.001 to about 100 mg of active compound per kilogram of body weight, administered orally to a mammalian patient. In another embodiment, the total daily dose range is between about 0.01 to about 20 mg of active compound per kilogram of body weight. In another embodiment, the total daily dose range is between 0.1 to about 10 mg of active compound per kilogram of body weight. If desired, the effective daily dose may be divided into multiple doses for purposes of administration, e.g. two to four separate doses per day.

Alternatively, the total daily dose range of the active ingredient of this invention is, in one embodiment, between about 1 and 500 mg per 70 kg of body weight per day; or, in another embodiment, about 10 and 500 mg per 70 kg of body weight per day; or, in another embodiment, between about 50 and 250 mg per 70 kg of body weight per day; or, in another embodiment, between about 100 and 150 mg per 70 kg of body weight per day.

It is intended herein that by recitation of such specified ranges, the ranges cited also include, in one embodiment, all those dose range amounts between the recited range. For example, in the range about 1 and 500, it is intended to encompass 2 to 499, 3-498, etc, without actually reciting each specific range. The actual preferred amounts of the active ingredient will vary with each case, according to the species of mammal, the nature and severity of the particular affliction being treated, and the method of administration.

It is also understood that doses within those ranges, but not explicitly stated, such as 30 mg, 50 mg, 75 mg, etc. are encompassed by the stated ranges, as are amounts slightly outside the stated range limits.

Alternatively, the total daily dose range of the PAF antagonist compound of this invention is, in one embodiment, between about 10⁻⁸ and 10⁻³ molar range per 70 kg of body weight per day, or, in another embodiment, about 10⁻⁷ and 10⁻⁴ molar range per 70 kg of body weight per day, or, in another embodiment, between about 10⁻⁶ and 10⁻² molar range per 70 kg of body weight per day, or, in another embodiment, between about 10⁻⁵ and 10⁻¹ molar range per 70 kg of body weight per day. It is intended herein that by recitation of such specified ranges, the ranges cited also include all those concentration amounts between the recited range. For example, in the range about 10⁻⁸ and 10⁻³ molar range, it is intended to encompass 1.1×10⁻⁸ to 9.9×10⁻⁴, 1.2×10⁻⁸ to 9.8×10⁻⁴, etc, without actually reciting each specific range. The actual preferred amounts of the active ingredient will vary with each case, according to the species of mammal, the nature and severity of the particular affliction being treated, and the method of administration.

The term “unit dose” is meant to describe a single dose, although a unit dose may be divided, if desired. Although any suitable route of administration may be employed for providing the patient with an effective dosage of the composition according to the methods of the present invention, oral administration is preferred. Suitable routes include, for example, oral, rectal, parenteral (e.g., in saline solution), intravenous, topical, transdermal, subcutaneous, intramuscular, by inhalation, and like forms of administration may be employed. Suitable dosage forms include tablets, troches, dispersions, suspensions, solutions, capsules, patches, suppositories, and the like, although oral dosage forms are preferred.

Useful dosages of the compounds of the present invention can be determined by comparing their in vitro activity, and in vivo activity in animal models. Methods for the extrapolation of effective dosages in mice, and other animals, to humans are known to the art; for example, see U.S. Pat. No. 4,938,949.

### EXAMPLE ONE

### Introduction

This study examined the effect of PAF and PAF analogs on the release of the pro-inflammatory mediator, prostaglandin E₂ (PGE₂), from rat cortical cell preparations enriched in astrocytes, an in vitro cell culture system that is a model for reactive astrocytes. PAF is readily hydrolyzed by extra- and intra-cellular PAF acetylhydrolases (PAF-AH); therefore a non-hydrolyzable analog of PAF, methylcarbamyl-PAF (mc-PAF) was used for some experiments. The synthetic PAF analogs PAF-16 and PAF-18; the PAF precursor lyso-PAF; and the structurally similar lipids phosphatidylcholine (PC) and lyso-phosphatidylcholine (lyso-PC) were also assessed, to better determine the mechanism of PAF action. Whether co-incubation of AA and mc-PAF could have a synergistic effect on PGE₂ release was also assessed. Finally, the potential site(s) of PAF action was investigated, by examining the effect of specific PAF binding site antagonists on the mc-PAF-induced PGE₂ release.

### Materials and Methods

### Cell culture

All experimental protocols were approved by the Massachusetts Institute of Technology institutional review committee and meet the guidelines of the National Institutes of Health. Dissociated astrocytes were cultured from cortices of postnatal day 1-2 rat pups (as described by K. D. McCarthy, et al., Preparation of separate astroglial and oligodendroglial cell cultures from rat cerebral tissue, J. Cell Biol. 85 (1980) 890-902, with minor modifications R. K. K. Lee, et al., Metabolic glutamate receptors increase amyloid precursor protein processing in astrocytes: inhibition by cyclic AMP, J. Neurochem. 68 (1997) 1830-1835.) In brief, cells from dissociated cortices were plated onto poly-L-lysine coated 35- or 100 mm culture dishes. The initial culture media, minimal essential medium (MEM, Gibco-Life Technologies; Rockville, Md.) containing 15% horse serum (BioWhittaker; Walkersville, Md.), were aspirated 2-5 h after plating to remove unattached cells and debris, and replaced with MEM containing 5% fetal bovine serum (FBS, BioWhittaker; Walkersville, Md.). Half the medium was replaced with MEM/5% FBS twice weekly. Astrocytes were kept at 37° C. in a humidified 5% CO₂/95% air incubator for 9-15 days, by which time the cultures were confluent and could be used for experiments.

Immunohistochemical procedures were carried out to more precisely identify the cell types in the cultures. Cells were fixed with 4% paraformaldehyde in 0.1 M phosphate buffered saline (PBS; pH 7.4) for 10 min, incubated in Chemiblock (Chemicon, Temecula, Calif.) solution for 1 h, and incubated with primary antibodies (CD-45, NF-145, NF-70 (1:1000) Calbiochem, La Jolla, Calif.), N-200 and GFAP (1:2,000 and 1:3000, respectively; Sigma, St. Louis, Mo.) overnight at room temperature on an orbital shaker. Cells were then incubated with a biotinylated secondary antibody for 30 min, followed by an incubation with ABC (Vector, Burlingtom, VR) solution for 30 min. Cells were then placed for 6 minutes in a 0.02% 3,3-diaminobenzadine tetrahydrochloride (DAB) solution containing H₂O₂ for visualization of the bound chromogen.

Most of the cells in this preparation (approximately 85% of cultured cells) were immunopositive for the astrocyte-specific intermediate filament protein glial fibrilary acidic protein (GFAP), and had the characteristics of flat type 1-like astrocytes. It should be noted, however, that endothelial cells might also be immunopositive for GFAP, (F. A. Ghazanfari, et al., Characteristics of endothelial cells derived from the blood-brain barrier and of astrocytes in culture, Brain Res 890 (2001) 49-65.)

The only other immunologically identifiable cells were microglia (approximately 5% of cultured cells are immunopositive for CD-45). No neurons were detected using neurofilament-specific antibodies. Many of the remaining cells exhibited a morphology reminiscent of radial glia that have not yet assumed the genetic program of mature astrocytes, (E. D. Laywell, et al., Identification of a multipotent astrocytic stem cell in the immature and adult mouse brain, Proc. Natl. Acad. Sci. 97 (2000) 13883-13888.)

### Drug Preparation

Mc-PAF (Biomol; Plymouth Meeting, PA) was dissolved in methanol at a stock concentration of 10 mM. PAF-16, PAF-18, lyso-PAF, AA (Cayman Chemicals), lyso-PC and PC (Sigma) were dissolved in ethanol at stock concentrations of 10 mM. All stock solutions of lipids were stored at −80° C. and were used within 6 weeks of reconstitution. BN 52021 and CV 6209 (Biomol) were dissolved in ethanol. These PAF antagonists were stored at −20° C. in stock concentrations of 100 mM. BN 50730 (Biomeasure; Milford, Mass.) was dissolved in 45% hydroxy-B-cyclodextrin (HBC). All agents were diluted in warned serum-free medium prior to cell stimulation. Equal amounts of vehicle was added to control cells.

### Drug Treatments

Cells used for all experiments were established in vitro 9-15 days prior to use in experiments and were over 95% confluent. Serum-containing media were changed every 3-4 days. Cells were serum-deprived 24 hours prior to experimental treatments. Where treatment with PAF antagonists is indicated, these compounds were added 30 minutes prior to the addition of other agents.

### PGE₂ Assay

PGE₂ levels were measured by ELISA according to manufacturer's instructions (Cayman Chemicals, Ann Arbor, Mich.). Since the amount of PGE₂ in fresh medium was negligible, (J. Luo, et al., Transforming growth factor B1 regulates the expression of cyclooxygenase in cultured cortical astrocytes and neurons, J. Neurochem. 71 (1998) 526-534), direct assays of the PGE₂ concentration in cell-conditioned medium was used as a measurement of PGE₂ secretion by cultured cells. Results were derived from at least 3 separate experiments, assayed in duplicate or triplicate (n=6-8). The reliable detection limit of this assay (i.e. sensitivity) averaged 14+/−6 pg of PGE₂.

### Statistical Analysis

Data were expressed as means+/−SEMs. Statistical analyses were performed using unpaired Student's t-tests or ANOVAs for comparisons between groups, followed by Fischer's PLSD post hoc comparisons by means contrast. P values <0.05 were considered statistically significant.

### Results

### Effect of Solvents on Astrocytic PGE₂ Release

Addition of either methanol or ethanol (or a combination of both) to astrocyte-enriched cortical cell cultures caused an increase in PGE₂ release (less than a 10% increase) that was not statistically significant; HBC had no effect on PGE₂ release (data not shown).

### mc-PAF Increases Astrocytic PGE2 Release in a Time-Dependent Manner

Addition of the non-hydrolyzable PAF analog mc-PAF (1 μM) to treatment media caused a time-dependent increase in PGE₂ release from astrocyte-enriched cortical cell cultures (FIG. 1). Within 5 minutes of mc-PAF incubation, an increase in PGE₂ release was observed (p<0.05). The maximum mobilization of PGE₂ occurred at 30 minutes (p<0.01), decreasing gradually by 4 hr. A second peak, albeit smaller, was observed at 8 hr (p<0.05), and levels returned to baseline by 12 hr. As the peak release of PGE₂ by mc-PAF occurred at 30 min, this time was used in subsequent studies to assess potential mechanisms of PAF-induced PGE₂ release.

### PAF Analogs Increase Astrocytic PGE₂ Release in a Concentration-Dependent Manner

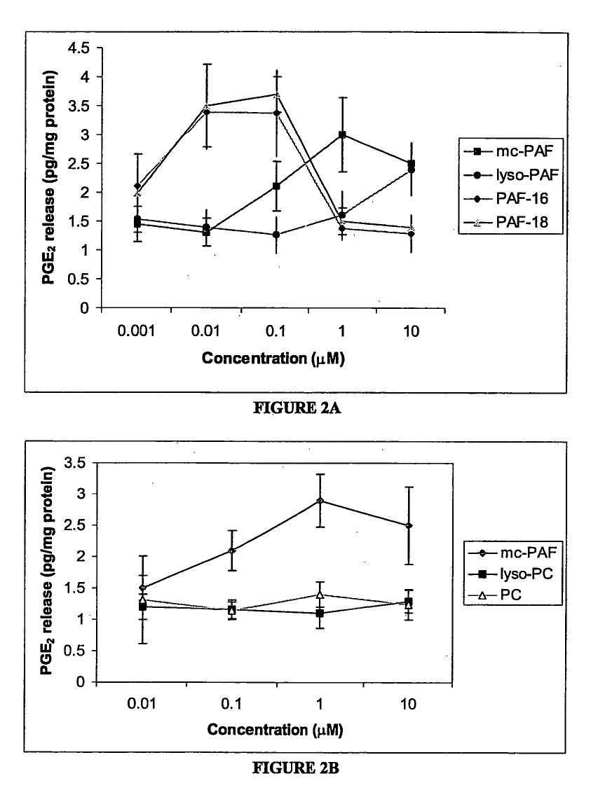

Addition of mc-PAF, lyso-PAF, PAF-16, or PAF-18 to astrocyte-enriched cortical cell cultures resulted in concentration-dependent increases in PGE₂ release into the conditioned media FIG. 2A). Mc-PAF significantly increased PGE₂ release at concentrations of 0.1 (p<0.05), 1 (p<0.01), and 10 (p<0.01) μM, and lyso-PAF at a concentration of 10 (p<0.05) μM. Both PAF-16 and PAF-18 increased PGE₂ release at concentrations of 0.01 (p<0.05), and 0.1 (p<01) μM, but were less effective at higher concentrations (FIG. 2A).

Though treatment with PAF-16 or PAF-18 caused significant effects, these effects were more variable across and within experiments than those produced by lyso-PAF or mc-PAF. For this reason, mc-PAF was used to explore the mechanisms mediating PAF-induced mobilization of PGE₂. Addition to the media of PC or lyso-PC, lipids, which are structurally similar to PAF analogs, had no effect on PGE₂ release at any concentration examined (10, 1, 0.1, and 0.01 μM; (FIG. 2B).

### Arachidonic Acid and mc-PAF Act Synergistically to Increase Astrocytic PGE₂ Release

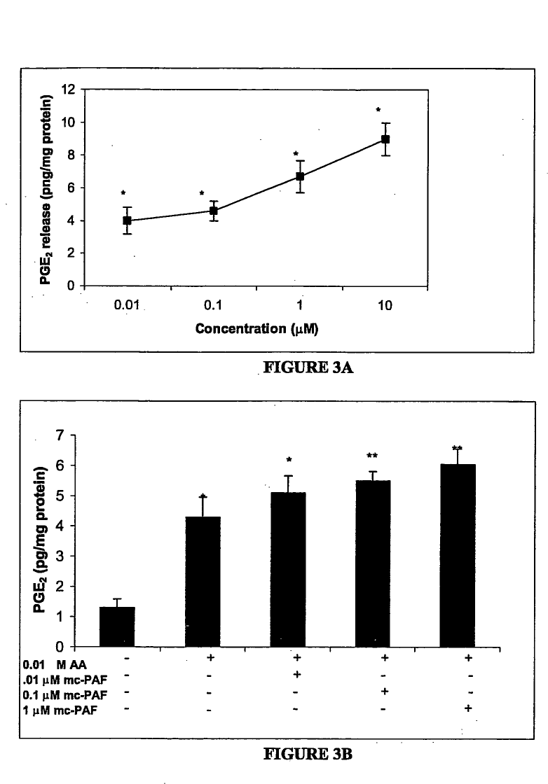

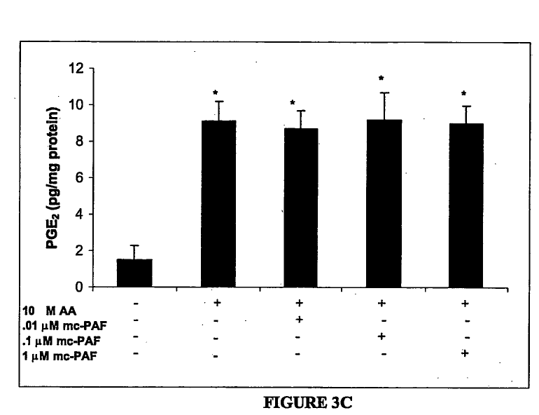

Treatment of astrocyte-enriched cell cultures for 30 minutes with AA (0.01-10 μM) increased PGE₂ release (p<0.01; FIG. 3A). Co-administration of AA with mc-PAF (0.1, 1 or 10 μM) caused an additive increase in PGE₂ release with a low arachidonate concentration (0.01 μM) (p<0.05; FIG. 3B), but not at a high AA concentration (10 μM) (FIG. 3C). These results suggest a “ceiling effect” might have blocked added responses to higher AA concentrations (i.e. no synergism), perhaps mediated by limits in the availability of downstream enzymes responsible for AA conversion to PGE₂ (e.g. cyclooxygenases).

### Effect of Intracellular PAF Binding Site Antagonists on PAF Analog- and AA-Induced PGE₂ Release

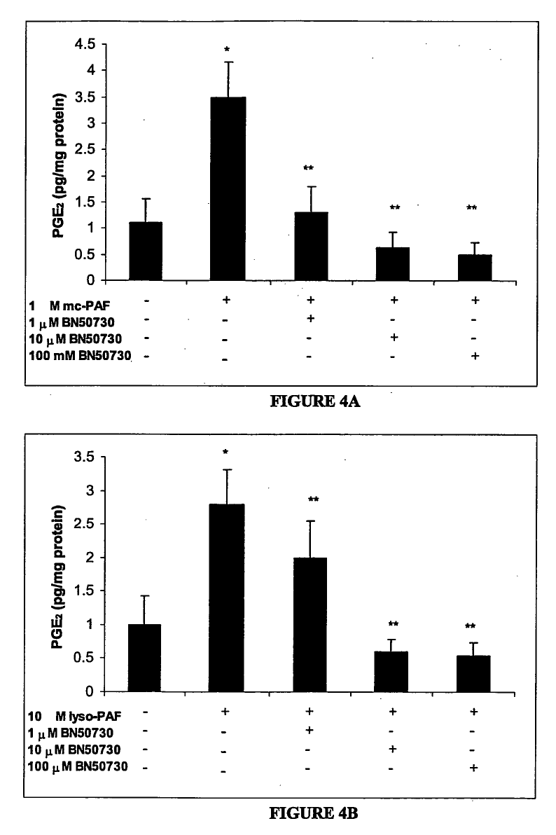

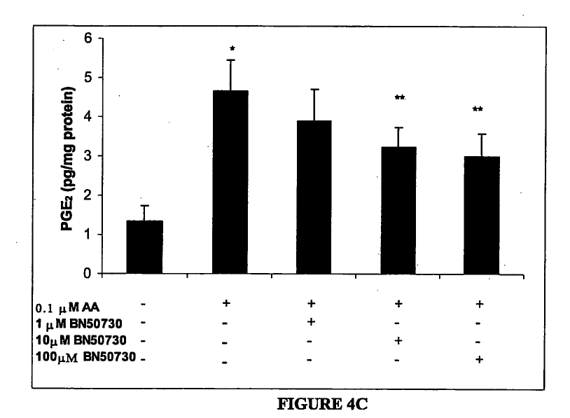

Prior exposure of cells to BN 50730 (0.1-100 μM) attenuated mc-PAF-induced PGE₂ release (FIG. 4A). Prior administration of BN 50730 also significantly attenuated the increase in PGE₂ release induced by lyso-PAF (FIG. 4B). These results demonstrate that intracellular PAF binding sites are necessary for the PAF analog-induced effect on PGE₂ mobilization. Prior exposure of cells to BN 50730 also significantly attenuated the release of PGE₂ induced by AA (FIG. 4C), showing that exogenous AA increases intracellular PAF.

### Effect of Cell Surface PAF Antagonists on PAF Analog- and AA-Induced PGE₂ Release

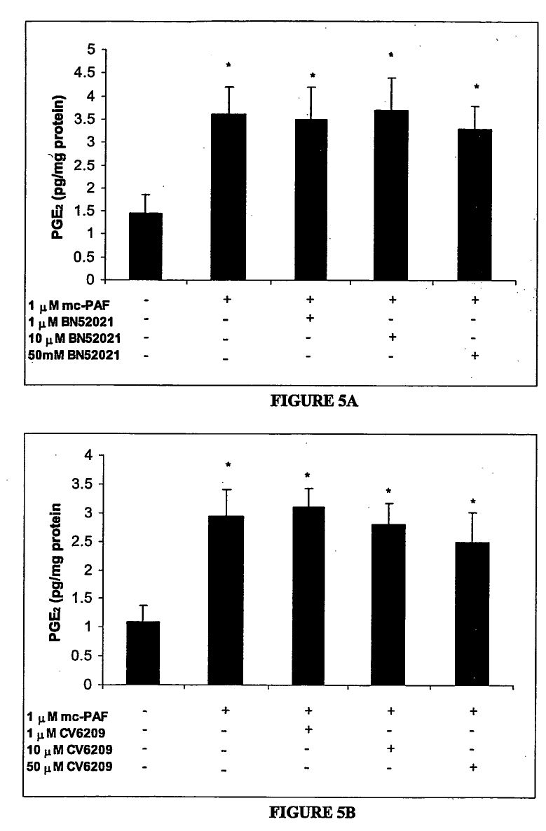

BN 52021 and CV 6209, two structurally distinct antagonists to cell surface PAF receptors, had no significant effect on mc-PAF-induced PGE₂ release (FIGS. 5A, 5B, respectively) at concentrations that effectively block the plasma membrane receptors. At higher concentrations both antagonists attenuated by 20-25% the mc-PAF-induced increase in PGE₂; this effect could have been caused by blockade at intracellular sites. These agents had no effect on the PGE₂ release caused by lyso-PAF-or AA (data not shown). When either BN 52021 or CV 6209 was administered alone (i.e. no mc-PAF), PGE₂ release was increased, perhaps by shunting endogenous PAF to intracellular binding sites.

### Discussion

These data show that PAF enhanced PGE₂ release from cortical astrocytes; that mc-PAF and lyso-PAF shared this effect; that related phosphatides (PC, lyso-PC) failed to enhance PGE₂ release; that AA synergized the effect of mc-PAF on PGE₂ production; and that intracellular PAF antagonists could attenuate the PGE₂ response elicited by PAF analogs and AA.

Increasing the concentration of mc-PAF (0.001-10 μM) caused an increase in the amounts of PGE₂ released into the media (FIG. 2A). The highest concentration of mc-PAF used in this study (10 μM) increases PGE₂ release, however greater variability was observed, with some cultures displaying no increases in PGE₂ release. Incubation of cells with this high concentration for 24 hours caused cytologic evidence of toxicity. As 1 μM mc-PAF does not appear to have toxic effects and produces a reliable PGE₂ increase that varies very little across cultures (relative to other concentrations), this concentration was used to explore the site of PAF action. In contrast to mc-PAF's concentration-response effect on PGE₂ release, peak PGE₂ release was observed with 0.1 μM PAF-16 or PAF-18, and higher and lower concentrations of these compounds elicited less release (FIG. 2A). While it is unlikely that cell death occurred within 30 min, it is possible that these higher concentrations elicited a cellular program distinct from the physiological program activated by lower concentrations. Also, higher concentrations of synthetic PAF might have resulted in poor solubility or extracellular micelle formation, causing less PAF to enter the cells.

PC and lyso-PC, which have similar abilities to perturb membranes, failed to affect PGE₂ release (FIG. 2B), suggesting that the PAF, mc-PAF and lyso-PAF effects were a result of specific actions on PAF binding sites, rather than non-specific membrane perturbation. As lyso-PAF does not activate cell surface PAF receptors, this lipid may have caused PGE₂ release by its conversion to intracellular PAF. Hydrolysis of lyso-PAF to lyso-PAF might prevent the lipids from reaching intracellular sites.

BN 50730, a competitive antagonist to intracellular PAF binding sites, prevented mc-PAF-induced PGE₂ release (FIG. 4A). Accumulation of PAF was accompanied by initial activation of cPLA₂ (within 5 min), followed by lyso-PAF-AT activation. These findings not only support a role for PAF in PGE₂ release, but also demonstrate that the enzyme responsible for lyso-PAF conversion to PAF is also activated early in LPS-induced PGE₂ release.

BN 50730 also attenuateed PGE₂ release induced by lyso-PAF (FIG. 4B) and AA (FIG. 4C). While BN 50730 completely abolished lyso-PAF and mc-PAF generated PGE₂ release, it only attenuated the AA-induced PGE₂ release. This shows that intracellular PAF binding sites were required for the effects of mc-PAF and lyso-PAF on PGE₂ release, but not for those of AA. As AA can induce PGE₂ release even when these intracellular PAF binding sites are blocked, these findings show that the sites were not necessary for PGE₂ production when exogenous AA is made available to cells. However, the blockade of intracellular PAF binding sites did attenuate some of the mobilization of PGE₂ by AA. This attenuation may be explained by other actions of exogenous AA on cells. For instance, AA increases cPLA₂ activation. Thus, besides providing the necessary substrate for PGE₂ synthesis, exogenous AA can also produce more AA (and PAF) by activating cPLA₂. It is of interest to note, that in the case of lyso-PAF and mc-PAF, higher concentrations of BN 50730 not only attenuate the PAF analog-induced PGE₂ release but also reduce the basal release of PGE₂, suggesting that endogenous intracellular PAF has a role in basal PGE₂ release.

CV-6209 and BN 52021, which are structurally distinct antagonists to PAF's plasma membrane receptors, did not significantly influence mc-PAF-induced PGE₂ release (FIGS. 5A and 5B). Administration of these agents alone increased PGE₂ release (data not shown). Not to be limited by theory, this effect may have been caused by a compensatory increase in PAF synthesis and/or a shunting of endogenously produced PAF to intracellular sites.

Marked increases in AA levels and eicosanoids (including PGE₂) have been observed in association with brain inflammation. It is thus proposed herein that the PAF-induced PGE₂ release initiates an inflammatory cascade in astrocytes that can be detrimental to central nervous system function.

### Summary

The phospholipid mediator platelet-activating factor (PAF) increased the release of prostaglandin E₂ (PGE₂) from astrocyte-enriched cortical cell cultures in a concentration- and time-dependent manner. The non-hydrolyzable PAF analog methylcarbamyl-PAF (mc-PAF), the PAF intermediate lyso-PAF, and arachidonic acid (AA) also produced this effect. In contrast, phosphatidlycholine (PC) and lyso-PC, lipids that are structurally similar to PAF and lyso-PAF, had no effect on PGE₂ production, demonstrating that PAF-induced PGE₂ release was not the consequence of non-specific phospholipid-induced membrane perturbation. Antagonism of intracellular PAF binding sites completely abolished the ability of mc-PAF and lyso-PAF to mobilize PGE₂, and attenuated the AA effect. Antagonism of the G-protein-coupled PAF receptor in plasma membranes had no significant effect on mc-PAF, lyso-PAF or AA-induced PGE₂ release. It is thus proposed that intracellular PAF is a physiologic stimulus of PGE₂ production in astrocytes.

### EXAMPLE TWO

### Introduction

The formalin test, a commonly used model of inflammatory nociception in rats, which elicits a biphasic behavioral response, (Dubuisson D, et al., The formalin test: a quantitative study of the analgesic effects of morphine, meperidine, and brain stimulation of rats and cats. Pain 4 (1977)161-174), was used to assess the involvement of PAF in nociception. The early phase starts immediately after injection of formalin, lasts about 5 min, and is thought to result from direct chemical stimulation of nociceptive fibers, (Jongsma et al., Markedly reduced chronic nociceptive response in mice lacking the PAC1 receptor. NeuroReport 12 (2001) 2215-2219). The late phase is exhibited 15-70 minutes after formalin injection and appears to depend on the combination of an inflammatory reaction in the peripheral tissue and functional changes in the dorsal horn of the spinal cord, (Tjolsen et al., The formalin test: an evaluation of the method. Pain 51 (1992) 5-17). To investigate the role of PAF in nociception, and the potential site(s) of its action, two structurally distinct PAF antagonists were administered systemically to rats 40 minutes prior to formalin injection, and their effects on the biphasic formalin response were measured. BN 52021 is a competitive PAF antagonist that selectively inhibits the cell surface PAF receptor, while BN 50730 is a specific inhibitor for intracellular PAF binding sites (Marcheselli et al., Distinct platelet-activating factor binding sites in synaptic endings and in intracellular membranes of rat cerebral cortex. J Biol Chem 265 (1990) 9140-9145; Marcheselli et al., Platelet-activating factor is a messenger in the electroconvulsive shock-induced transcriptional activation of c-fos and zif-268 in hippocampus. J Neurosci Research 37 (1994) 54-61.

### Materials and Methods

### Animals

Sixty male Sprague Dawley rats weighing 300-350 g (Taconic Labs, Canada) were housed in groups of 2-3 per cage, in polycarbonate cages. Animals were maintained under standard environmental conditions (room temperature: 20-20° C.; relative humidity: 55-60%; light/dark schedule: 12/12 hr) with free access to standard laboratory chow and tap water.

### Drug Preparation and Administration

BN 50730 (Biomeasure; Milford, Mass.) and BN 52021 (Biomol) were dissolved in 45% hydroxypropyl-B-cyclodextrin in distilled water (HBC). Drugs (at doses of 10, 1, or 0.1 mg/kg) or vehicle were administered intraperitoneally (i.p.) 40 minutes prior to formalin injection. The doses chosen were based on those found to produce central effects after their peripheral administration.

### Formalin Test

Experiments were carried out in accordance with The National Institutes of Health Guide for the Care and Use of Laboratory Animals. Behavioral testing was carried out in a blind manner. Nociceptive responses were examined in the formalin test described previously, (Dubisson et al., The formalin test: a quantitative study of the analgesic effects of morphine, meperidine, and brain stimulation of rats and cats. Pain 4 (1977) 161-174). In brief, animals were placed in a clear Plexiglas® formalin test box (30 cm×30 cm×30 cm), with a mirror positioned at a 45° angle below the floor allowing for unobstructed observation of the animal's paw. Following a 10-min habituation period, animals were removed from the formalin box, at which time 50 μl of 1% formalin was injected subcutaneously (s.c.) into the plantar surface of the right hind paw with a 27-gauge needle. The amount of time that each rat elevated the injected paw was recorded in five-min intervals during the 70-min period following formalin injection. Each animal was used once.

The 60-min formalin test produced a biphasic response consisting of an initial, rapidly decaying acute phase (early phase, 1-10 minutes after injection) followed by a slow rising and long-lived tonic phase (late phase, 15-60 minutes after injection). Typically animals elevated their paws following injection (i.e. the early phase) followed by a reduction in this behavior. Approximately 15-20 minutes after injection, the inflammatory late phase began, and animals again elevated their paws to varying degrees for the remainder of the testing period. The amount of time animals elevated their injected paw was used as a behavioral measure of pain.

### Data Analysis

Data were expressed as means+/−SEM, and p values <0.05 were considered statistically significant. Treatment groups were compared with vehicle-controls using one-way analysis of variance (ANOVA) followed by Fischer's PLSD post-hoc test to compare between groups if overall significance was found by ANOVA.

### Results

### BN 52021 Effects on Formalin-Induced Nociception

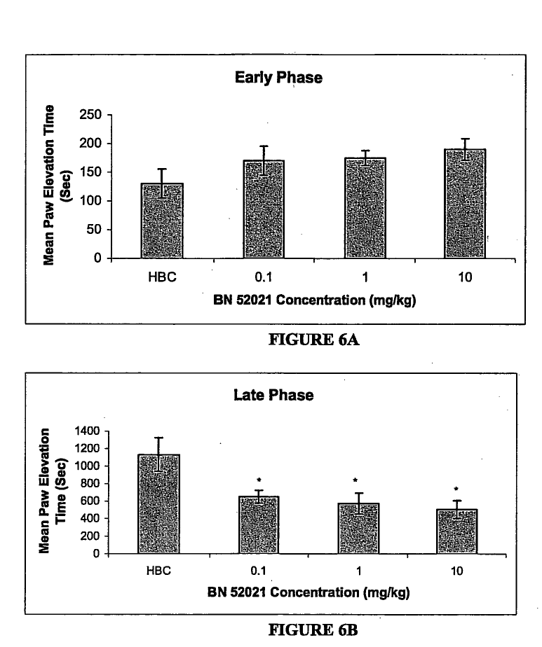

The nociceptive response (measured as time spent with the injected paw elevated) during the early phase (1-10 minutes post-formalin) was not significantly affected by BN 52021 administration (FIG. 6A) although rats that received BN 52021 tended to elevate their paws for longer periods of time than did vehicle-treated controls. During the late phase (10-60 min), BN 52021-treated rats elevated their paws for significantly shorter times than do control-treated rats [F(3,30)=3.831, p<0.05; FIG. 1B]. Fisher's PLSD post-hoc analysis revealed that the responses of rats receiving 10 (p=0.008), 1 (p=0.013) or 0.1 (p=0.0366) mg/kg BN 52021 differed significantly from those of control-treated rats.

### BN 50730 Effects on Formalin-Induced Nociception

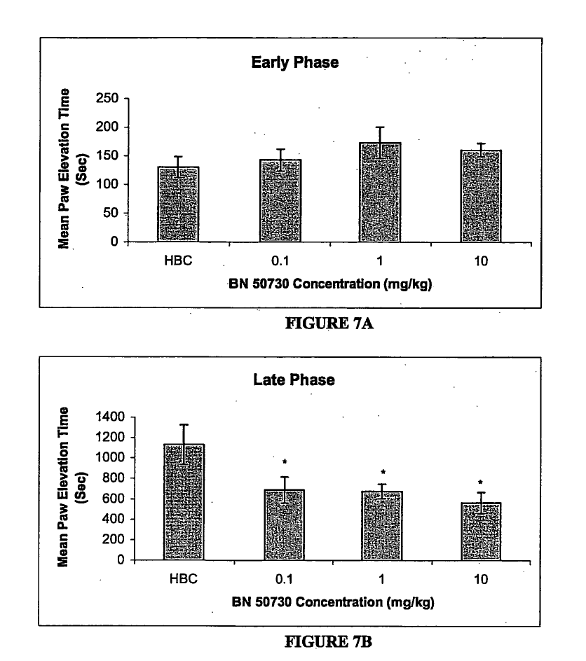

The nociceptive response (measured as time spent with the injected paw elevated) during the early phase (1-10 minutes post-formalin) was not significantly affected by BN 50730 administration (FIG. 7A). During the late phase (10-60 min), BN 50730-treated rats exhibited significantly shorter paw elevation times than did control-treated rats [F(3,30)=2.933, p<0.05; FIG. 6B]. Fisher's PLSD post-hoc analysis revealed that the behavior of rats receiving 10 (p=0.016), 1 (p=0.046) or 0.1 (p=0.049) mg/kg BN 50730 differed significantly from those of control-treated rats.

### Discussion

These data show that systemic administration of PAF antagonists, which act selectively on cell surface or intracellular PAF binding sites (BN 52021 and BN 50730, respectively), decreased nociceptive behavior during the late, but not the early, phase of the formalin test in rats (FIGS. 6B and 7B). Although three doses were used for each antagonist, a dose-response relationship was not revealed for either drug (i.e. all three doses of BN 52021 and BN 50730 decrease nociceptive behavior in a similar fashion). These results demonstrate the role of endogenous PAF in nociception. PAF is extremely potent and tightly regulated; the lowest dose of each antagonist is likely sufficient to have blocked enough of the the binding sites to have prevented endogenous PAF from carrying out it's nociceptive function(s) at both intracellular and plasma membrane sites.

A feature of the formalin test in rodents is that animals show two distinct phases of nociceptive behavior, which seem to depend on different mechanisms, (Dubisson et al., The formalin test: a quantitative study of the analgesic effects of morphine, meperidine, and brain stimulation of rats and cats. Pain 4 (1977) 161-174). Substance P and bradykinin participate in the early phase, while histamine, serotonin and prostanoids appear to be involved in the late phase, (Shibata et al., Modified formalin test: characteristic pain response. Pain 29 (1989) 375-386). The early phase of formalin-induced nociception (also known as the acute phase) starts immediately after its injection, and is thought to result from direct chemical stimulation of chemosensitive nociceptors, (Dubisson et al., The formalin test: a quantitative study of the analgesic effects of morphine, meperidine, and brain stimulation of rats and cats. Pain 4 (1977) 161-174; Hatakeyama et al., Differential nociceptive responses in mice lacking the α1B subunit of N-type Ca²⁺ channels. NeuroReport 12 (2001) 2423-2427; Jongsma et al., Markedly reduced chronic nociceptive response in mice lacking the PAC1 receptor. NeuroReport 12 (2001) 2215-2219). The second phase (also known as the tonic phase) is thought to result from peripheral inflammatory processes, and from sensitization in the spinal cord produced by the first phase, (Tjolsen et al., The formalin test: an evaluation of the method. Pain 51 (1992) 5-17), as well as from functional changes in central processing, (Coderre et al., Central nervous system plasticity in the tonic pain response to subcutaneous formalin injection. Brain Res 535 (1990) 155-158). As both antagonists tend to increase nociceptive responses (albeit not significantly, FIGS. 6A and 7A) during the early phase, the decrease in nociceptive responses during the late phase cannot be attributed to a reduction in the early phase of formalin-induced nociception.

In conclusion, the nociceptive responses to subcutaneous formalin injection were significantly reduced in rats receiving PAF antagonists that act on intracellular or cell surface PAF binding sites, demonstrating that selective PAF antagonists are effective in the treatment of certain forms of acute and chronic pain.

### Summary

Platelet-activating factor (PAF) is a membrane-derived phospholipid mediator that has biological effects on a variety of cells and tissues. A variety of stimuli, including those producing inflammation, promote the synthesis and release of PAF from various cell types. Evidence suggests that PAF exerts cellular actions through a plasma membrane receptor as well as via intracellular (microsomal) PAF binding sites. This second example: 1) investigated the role of PAF in a model of inflammatory nociception in rats (i.e. the formalin test), and 2) localized PAF's site(s) of action in nociception. The effect of administering two PAF antagonists (BN 52021 and BN 50730, which are selective for cell surface and intracellular PAF binding sites, respectively) was assessed on formalin-induced nociceptive responses. Forty minutes prior to formalin injection into the rat hindpaw, male Sprague Dawley rats received systemic injections of BN 52021 (10, 1, or 0.1 mg/kg), BN 50730 (10, 1, or 0.1 mg/kg), or vehicle (45% 2-hydroxypropyl-B-cyclodextrin in distilled water, HBC) and the effects of the drugs on nociceptive behavioral responses were measured. Rats receiving systemic BN 52021 or BN 50730 displayed a significant reduction of nociceptive responses in the late, but not early, phase of formalin-induced nociception. These findings demonstrate a role for endogenous PAF in nociceptive transmission, especially for persistent pain like that which occurs in the late phase of the formalin test. The findings also indicate that both intracellular and cell surface PAF binding sites are involved in nociceptive modulation in rats, and that PAF antagonists are useful for treating some forms of acute or chronic pain.

### EXAMPLE THREE

### Materials and Methods

### Drug Preparation

Mc-PAF (Cayman Chemical, Ann Arbor, Mich.) was dissolved in ethanol at a stock concentration of 10 mM. Indomethacin, piroxicam, NS-398 (Biomol; Plymouth Meeting, Mass.), and SC-560 (Cayman Chemical) were dissolved in 45% hydroxy-β-cyclodextrin (HBC; Sigma, St. Louis, Mo.). Cells were serum-deprived for 24 hrs prior to experimental treatments to induce quiescence. Where treatment with inhibitors is indicated, these compounds were added 30 min prior to the addition of mc-PAF.

### PGE₂ Assay

Direct assay of the PGE₂ concentration in cell-conditioned medium was used as an index of PGE₂ secretion by primary astrocytes. PGE₂ levels were measured by ELISA according to manufacturer's instructions (Cayman Chemicals, Ann Arbor, Mich.), as described.

### Data Analysis

Results were derived from at least 3 separate experiments, assayed in duplicate or triplicate (n=6−8). Data were expressed as means+/−SEMs. Statistical analyses were performed using ANOVAs for comparisons between groups, followed by Fischer's PLSD post hoc comparisons by means contrast. p values <0.05 were considered statistically significant.

### Results

### Effect of mc-PAF on PGE₂ Release

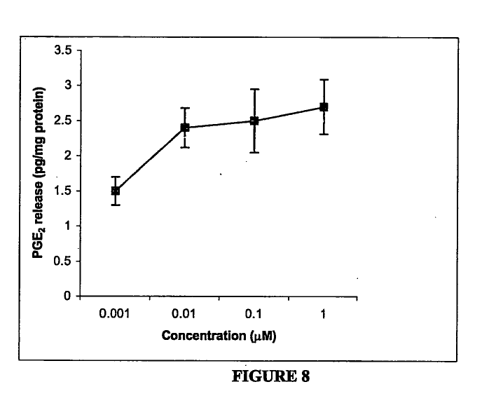

Addition of mc-PAF (0.001 to 1 μM) to the astrocyte-enriched cortical cell cultures resulted in concentration-dependent increases in the release of PGE₂ into the conditioned media (FIG. 8). As these primary astrocytes express both COX-1 and COX-2 according to Western blot analyses (data not shown), the involvement of each isozyme in the PAF effect was next assessed.

### Effect of Exposure to Piroxicam Plus SC-650

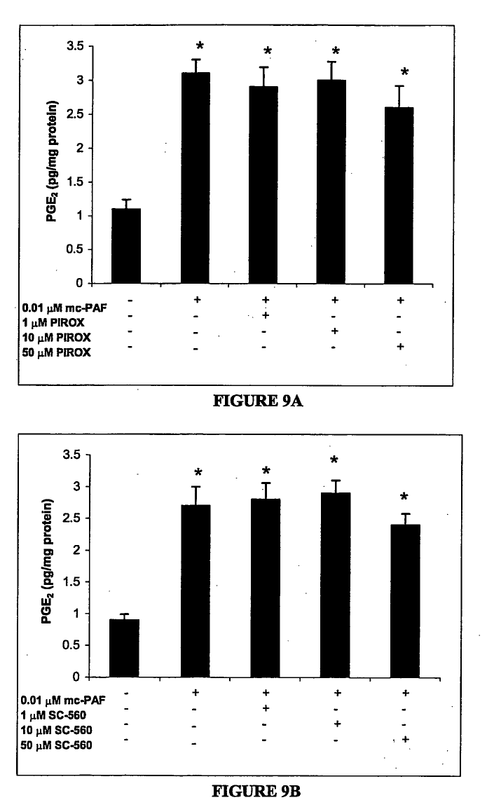

Prior exposure of cells to lower concentrations (1 or 10 μM of piroxicam (which is considered to be more specific for COX-1 than for COX-2; [Mitchell, J. A., Akarasereenont, P., Thiemermann, C., Flower, R. J. and Vane, J. R., Selectivity of nonsteroidal anti-inflammatory drugs as inhibitors of constituitve and inducible cyclooxygenase, Proc. Natl. Acad. Sci., 90 (1994) 11693-11697]) had no effect on mc-PAF-induced PGE₂ release (FIG. 9A). A higher concentration (50 μM) attenuated some of this PGE₂ release; this effect was not statistically significant. The COX-1 selective inhibitor SC-560 similarly did not significantly influence mc-PAF-induced PGE₂ release (FIG. 9B). These results demonstrate that COX-1 activity was not required for PAF-mediated PGE₂ release from astrocytes, even though COX-1 is expressed in these cells.

### Effect of Exposure to Indomethacin Plus NS398

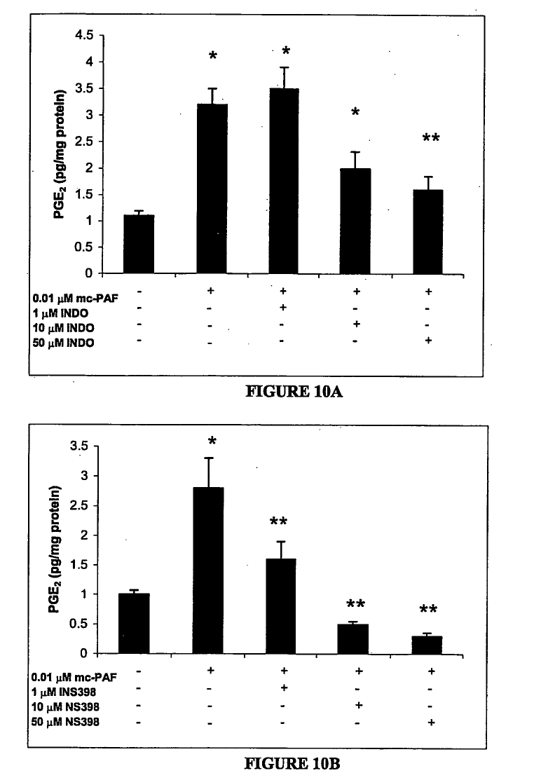

Prior exposure of astrocytes to the non-selective COX inhibitor indomethacin [Meade, E. A., Smith, W. L. and Dewitt, D., Differential inhibition of prostaglandin endoperoxide synthase (cyclooxygenase) isozymes by aspirin and other non-steroidal anti-inflammatory drugs, J. Biol. Chem., 268 (1993) 6610-6614] (1, 10, and 50 μM) attenuated the mc-PAF-induced PGE₂ release in a concentration-dependent manner without affecting basal PGE₂ release (FIG. 10A). The COX-2 selective inhibitor NS-398 [Masferrer, J. L., Zweifel, B. S., Manning, P. T., Hauser, S. D., Leahy, KM., Smith, W. G., Isakson, P. C. and Seiber, K., Selective inhibition of inducible cyclooxygenase 2 in vivo is antiiinflammatory and non-ulceogenic, Proc. Natl. Acad. Sci. (1994) 3228-3232] completely abolished mc-PAF-induced PGE₂ release (FIG. 10B); highest concentrations (10 and 50 μM) also prevented basal PGE₂ release. These results show that the COX-2 isozyme is required for PAF-induced PGE2 release from astrocytes.

### Discussion

Cells to have ample basal capacity for COX-catalyzed formation of PGE₂ by expressing either COX-1 or COX-2, or both. The present invention shows that COX-1 and COX-2 protein levels did not increase within 30 min of mc-PAF stimulation (as assessed by immunocytochemical and Western blot analyses; data not shown). Second, pre-treatment with either a transcription inhibitor (actinomycin D; 5 μg/ml) or a protein translation inhibitor (cyclohexamide; 10 μg/ml) had no effect on mc-PAF-induced PGE₂ release (data not shown). This findings show that (a) basal expression of COX-2 appears to be sufficient to sustain the PAF-induced response; and (b) pro-inflammatory stimuli can induce the de novo synthesis of COX-2 protein for PAF-induced astrocytic PGE₂ release in astrocytes.

The results of this study show that both PAF-induced and constitutive PGE₂ release are predominantly mediated by COX-2 in astrocytes; and that astrocytes express sufficient basal COX-2 activity to mediate the acute inflammatory response to PAF.

COX-2 is the major enzyme responsible for PG production in developing brain, and astrocytes are an important source of PGE₂ in developing brain [Peri, K. G., Hardy, P., Li, D. Y., Varma, D. R. and Chemtob, S., Prostaglandin G/H synthase-2 is a major contributor of brain prostaglandins in the newborn, J. Biol. Chem., 270 (1995) 24615-24620]. The present findings with cell cultures from early post-natal (1-2 days of age) rats show that COX-2 causes synthesis of astrocytic PGs early in development; indeed PAF-mediated PGE₂ release from astrocytes may have a role in development. Cultured astrocytes express elements of a reactive phenotype in culture, including COX-2 expression, and may thus provide a model for the activated astrocytes seen in various neurodegenerative and inflammatory-associated disorders. While glial activation can be protective, excess activation can be deleterious. In fact, activated astrocytes are neurotoxic in culture systems and may be involved in neurodegeneration in vivo. Moreover, PGE₂ release has been shown to induce neuronal degeneration. The present findings show that PAF impacts inflammatory-immune function of astrocytes by affecting COX-2-mediated PGE₂ release, and plays a role in inflammatory-immune-associated diseases.

### Summary

The phospholipid mediator platelet-activating factor (PAF) and its non-hydrolyzable analog methylcarbamyl-PAF (mc-PAF) increase prostaglandin E₂ (PGE₂) release from astrocyte-enriched cortical cell cultures. In this study the involvement of the COX isoforms in PAF-induced PGE₂ release was examined. Treatment of cells with the non-specific COX inhibitor indomethacin, or the specific COX-2 inhibitor NS-398, prior to mc-PAF stimulation completely blocked the PAF-induced release of PGE₂; treatment with more selective COX-1 inhibitors (i.e. piroxicam and SC-560) did not significantly do so. These data show that COX-2 is responsible for PAF-mediated PGE₂ release in primary astrocytes.

### EXAMPLE FOUR

### Antagonists of Cell-Surface paf Receptors Function in the Brain, While Antagonists of Intracellular paf Receptors Function in the Spinal Cord

### Materials and Methods

Animals were anesthetized (50 mg/kg sodium pentobarbitol), and unilateral guide cannulae (23 gauge) were implanted into the lateral ventricle. The guide cannulae were attached to the skull using jeweler's screws and dental acrylic. After surgery, stylets were inserted and left in place to ensure cannula patency. The formalin test was conducted 7-10 days post-surgery.

In other experiments, in which the antagonist was injected into the left hippocampus, coordinates for the guide cannulae were: AP=−3.1 mm, ML=1.5 mm from bregma, and DV=−2.0 mm from the skull surface.

The day prior to formalin testing, rats were placed in the boxes for a 15 min habituation period. The day of testing, vehicle or PAF antagonists were administered and the rats were placed in the boxes for 20 min. At this time, animals were removed from the box, and 50 μl of 1% formalin (0.4% formaldehyde) was injected subcutaneously into the plantar surface of the right hind paw with a 27-gauge needle. Immediately after injection, each animal was exposed to the open field box for 60 min and the amount of time they elevated their injected paw was recorded as described for previous Examples.

Upon completion of testing, animals were overdosed with sodium pentobarbitol and perfused with saline followed by 10% formalin solution. Brains were removed, fixed, and cut into 20-μm coronal sections throughout the cannula tract. Sections were then mounted, stained with cresyl violet, and coverslipped. Slides were examined using light microscopy for verification of injection needle tip location using the atlas of Paxinos and Watson (1986). The behavioral data for 4 rats were discarded from the study due to incorrect cannulae placement.

Data were expressed as means+/−SEM and p values <0.05 were considered statistically significant. Experimental groups were compared using a one-way analysis of variance (ANOVA) with repeated measures (5 min blocks of time) followed by Scheffe's post-hoc test. Independent t-tests were used to assess the effects of the PAF antagonists on the individual 5 min bins of time post-formalin as well.

### Results

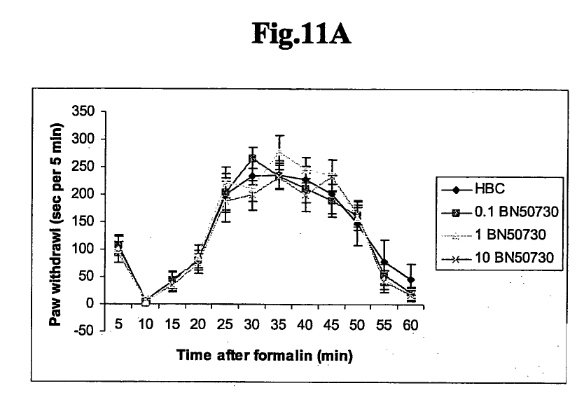

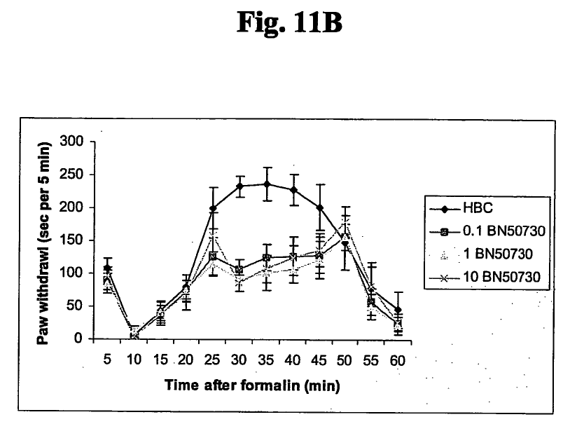

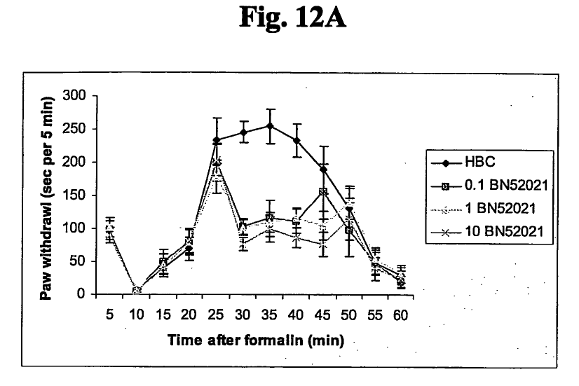

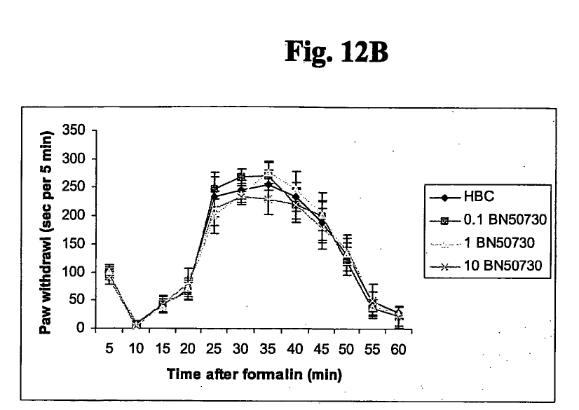

The late-phase of the nociceptive response was significantly affected by administration of BN 52021 into the lateral ventricle (FIG. 11A). ANOVA analysis indicated a significant main effect of time [F(11,25)=4.852, p<0.001], as would be expected considering the dynamic nature of the biphasic response. Moreover, a main effect of group was also revealed [F(3,25)=9.124, p<0.001]. Scheffe's post-hoc analysis indicated that the nociceptive responses of rats receiving 10 (p=0.001) or 1 (p=0.005) μg BN 52021 were significantly diminished compared with those of control-treated rats. Independent t-tests indicated that rats receiving the 10 and 1 μg concentrations of BN 52021 had significantly attenuated levels of paw elevation between 30 and 45 min post-formalin; the 1 μg concentration also exhibited decreased paw elevation at 50 min post-formalin.

The nociceptive response in rats was not significantly affected by BN 50730 administration into the lateral ventricle (FIG. 11B), as ANOVA analysis indicated no significant group effect [F(3,27)=1.29, p=n.s]. There was a significant main effect of time [F(11,27)=11.86, p<0.0001], due to the dynamic nature of the biphasic nociceptive response. Similar results were seen with intra-hippocampal administration of BN 52021 and BN 50730.

Thus, intra-hippocampal injection of BN 52021, but not BN 50730, decreases nociceptive behavior during the tonic or late phase of the formalin test, showing that cell surface, but not intracellular, PAF binding sites mediate inflammatory-based nociception in the brain.

By contrast, when PAF inhibitors were injected intrathecally (into the spinal cord), BN 50730, but not BN 52021, decreased the nociceptive response (FIG. 11 A-B). These findings show that intracellular, but not cell surface, PAF binding sites mediate inflammatory-based nociception in the spinal cord.

1. A method of treating pain, comprising blocking or inhibiting a cell surface platelet-activating factor (PAF) receptor, wherein said cell surface PAF receptor is in the brain.

2. The method of claim 1, wherein said blocking or inhibiting comprises administering BN 52021.

3. The method of claim 1, wherein said blocking or inhibiting comprises administering CV-3988, CV-3938, CV-6209, TCV-309, E5880, SRI 63441, ginkgolide A, ginkgolide B, ginkgolide C, ginkgolide T, ginkgolide M, or a derivative thereof, either alone or in combination.

4. The method of claim 1, wherein said blocking or inhibiting comprises administering a nutritional supplement, said nutritional supplement comprising Gingko biloba, or derivatives or constitutents thereof.

5. The method of claim 1, wherein said cell surface PAF receptor is in the cerebellum or the hippocampus.

6. The method of claim 1, wherein said blocking or inhibiting comprises administering a pharmaceutical composition or nutritional supplement to the lateral ventricle or the hippocampus.

7. A method of treating pain, comprising blocking or inhibiting an intracellular platelet-activating factor (PAF) receptor, wherein said intracellular PAF receptor is in the spinal cord.

8. The method of claim 7, wherein said blocking or inhibiting comprises administering a pharmaceutical composition, said pharmacetical composition comprising BN 50730.

9. The method of claim 8, wherein said blocking or inhibiting comprises administering a pharmaceutical composition, said pharmacetical composition comprising a pharmaceutically acceptable carrier and a therapeutically effective amount of a benzodiazapine or a derivative thereof, either alone or in combination.

10. The method of claim 8, wherein said blocking or inhibiting comprises administering a pharmaceutical composition, said pharmacetical composition comprising a pharmaceutically acceptable carrier and a therapeutically effective amount of WEB-2086, WEB-2170, Y-24180, BN 50727, BN 50730, BN 50739, and E 6123, SM-12502, YM264, ABT-299, SR 27417, SRI 63-073, ONO-6240, RO-19 3704, UK-74,505, BB-882, or a derivative thereof, either alone or in combination.

11. A method of treating inflammation, comprising blocking or inhibiting a cell surface platelet-activating factor (PAF) receptor, wherein said cell surface PAF receptor is in the brain.

12. The method of claim 11, wherein said blocking or inhibiting comprises administering a pharmaceutical composition, said pharmacetical composition comprising a pharmaceutically acceptable carrier and BN 52021.

13. The method of claim 11, wherein said blocking or inhibiting comprises administering a pharmaceutical composition, said pharmacetical composition comprising a pharmaceutically acceptable carrier and a therapeutically effective amount of CV-3988, CV-3938, CV-6209, TCV-309, E5880, SRI 63441, ginkgolide A, ginkgolide B, ginkgolide C, ginkgolide T, ginkgolide M, or a derivative thereof, either alone or in combination.

14. The method of claim 11, wherein said blocking or inhibiting comprises administering a nutritional supplement, said nutritional supplement comprising Gingko biloba, or derivatives or constitutents thereof.

15. The method of claim 11, wherein said cell surface PAF receptor is in the cerebellum or the hippocampus.

16. The method of claim 11, wherein said blocking or inhibiting comprises administering a pharmaceutical composition or nutritional supplement to the lateral ventricle or the hippocampus.

17. A method of treating inflammation, comprising blocking or inhibiting an intracellular platelet-activating factor (PAF) receptor, wherein said intracellular PAF receptor is in the spinal cord.

18. The method of claim 17, wherein said blocking or inhibiting comprises administering a pharmaceutical composition, said pharmacetical composition comprising BN 50730.

19. The method of claim 17, wherein said blocking or inhibiting comprises administering a pharmaceutical composition, said pharmacetical composition comprising a pharmaceutically acceptable carrier and a therapeutically effective amount of a benzodiazapine or a derivative thereof, either alone or in combination.

20. The method of claim 17, wherein said blocking or inhibiting comprises administering a pharmaceutical composition, said pharmacetical composition comprising a pharmaceutically acceptable carrier and a therapeutically effective amount of WEB-2086, WEB-2170, Y-24180, BN 50727, BN 50730, BN 50739, and E 6123, SM-12502, YM264, ABT-299, SR 27417, SRI 63-073, ONO-6240, RO-19 3704, UK-74,505, BB-882, or a derivative thereof, either alone or in combination.

21. A method of inhibiting a contraction of a uterus in a subject, comprising blocking or inhibiting a cell surface platelet-activating factor (PAF) receptor, wherein said cell surface PAF receptor is in the brain.

22. The method of claim 21, wherein said blocling or inhibiting comprises administering a pharmaceutical composition, said pharmacetical composition comprising a pharmaceutically acceptable carrier and BN 52021.

23. The method of claim 21, wherein said blocking or inhibiting comprises administering a pharmaceutical composition, said pharmacetical composition comprising a pharmaceutically acceptable carrier and a therapeutically effective amount of CV-3988, CV-3938, CV-6209, TCV-309, E5880, SRI 63-441, ginkgolide A, ginkgolide B, ginkgolide C, ginkgolide T, ginkgolide M, or a derivative thereof, either alone or in combination.

24. The method of claim 21, wherein said blocking or inhibiting comprises administering a nutritional supplement, said nutritional supplement comprising Gingko biloba, or derivatives or constitutents thereof.

25. The method of claim 21, wherein said cell surface PAF receptor is in the cerebellum or the hippocampus.

26. The method of claim 21, wherein said blocking or inhibiting comprises administering a pharmaceutical composition or nutritional supplement to the lateral ventricle or the hippocampus.

27. A method of inhibiting a contraction of a uterus in a subject, comprising blocking or inhibiting an intracellular platelet-activating factor (PAF) receptor, wherein said intracellular PAF receptor is in the spinal cord.

28. The method of claim 27, wherein said blocking or inhibiting comprises administering a pharmaceutical composition, said pharmacetical composition comprising BN 50730.

29. The method of claim 27, wherein said blocking or inhibiting comprises administering a pharmaceutical composition, said pharmacetical composition comprising a pharmaceutically acceptable carrier and a therapeutically effective amount of a benzodiazapine or a derivative thereof, either alone or in combination.

30. The method of claim 27, wherein said blocking or inhibiting comprises administering a pharmaceutical composition, said pharmacetical composition comprising a pharmaceutically acceptable carrier and a therapeutically effective amount of WEB-2086, WEB-2170, Y-24180, BN 50727, BN 50730, BN 50739, and E 6123, SM-12502, YM264, ABT-299, SR 27417, SRI 63-073, ONO-6240, RO-19 3704, UK-74,505, BB-882, or a derivative thereof, either alone or in combination.

31. A method of inhibiting a proliferation of a tumor cell in a subject, comprising blocking or inhibiting a cell surface platelet-activating factor (PAF) receptor, wherein said cell surface PAF receptor is in the brain.

32. The method of claim 31, wherein said blocking or inhibiting comprises administering a pharmaceutical composition, said pharmacetical composition comprising a pharmaceutically acceptable carrier and BN 52021.

33. The method of claim 31, wherein said blocking or inhibiting comprises administering a pharmaceutical composition, said pharmacetical composition comprising a pharmaceutically acceptable carrier and a therapeutically effective amount of CV-3988, CV-3938, CV-6209, TCV-309, E5880, SRI 63-441, ginkgolide A, ginkgolide B, ginkgolide C, ginkgolide T, ginkgolide M, or a derivative thereof, either alone or in combination.

34. The method of claim 31, wherein said blocking or inhibiting comprises administering a nutritional supplement, said nutritional supplement comprising Gingko biloba, or derivatives or constitutents thereof.

35. The method of claim 31, wherein said cell surface PAF receptor is in the cerebellum or the hippocampus.

36. The method of claim 31, wherein said blocking or inhibiting comprises administering a pharmaceutical composition or nutritional supplement to the lateral ventricle or the hippocampus.

37. A method of inhibiting a proliferation of a tumor cell in a subject, comprising blocking or inhibiting an intracellular platelet-activating factor (PAF) receptor, wherein said intracellular PAF receptor is in the spinal cord.

38. The method of claim 37, wherein said blocking or inhibiting comprises administering a pharmaceutical composition, said pharmacetical composition comprising BN 50730.

39. The method of claim 37, wherein said blocking or inhibiting comprises administering a pharmaceutical composition, said pharmacetical composition comprising a pharmaceutically acceptable carrier and a therapeutically effective amount of a benzodiazapine or a derivative thereof, either alone or in combination.

40. The method of claim 37, wherein said blocking or inhibiting comprises administering a pharmaceutical composition, said pharmacetical composition comprising a pharmaceutically acceptable carrier and a therapeutically effective amount of WEB-2086, WEB-2170, Y-24180, BN 50727, BN 50730, BN 50739, and E 6123, SM-12502, YM264, ABT-299, SR 27417, SRI 63-073, ONO-6240, RO-19 3704, UK-74,505, BB-882, or a derivative thereof, either alone or in combination.

41. A method of inhibiting angiogenesis in a subject, comprising blocking or inhibiting a cell surface platelet-activating factor (PAF) receptor, wherein said cell surface PAF receptor is in the brain.

42. The method of claim 41, wherein said blocking or inhibiting comprises administering a pharmaceutical composition, said pharmacetical composition comprising a pharmaceutically acceptable carrier and BN 52021.

43. The method of claim 41, wherein said blocking or inhibiting comprises administering a pharmaceutical composition, said pharmacetical composition comprising a pharmaceutically acceptable carrier and a therapeutically effective amount of CV-3988, CV-3938, CV-6209, TCV-309, E5880, SRI 63-441, ginkgolide A, ginkgolide B, ginkgolide C, ginkgolide T, ginkgolide M, or a derivative thereof, either alone or in combination.

44. The method of claim 41, wherein said blocling or inhibiting comprises administering a nutritional supplement, said nutritional supplement comprising Gingko biloba, or derivatives or constitutents thereof.

45. The method of claim 41, wherein said cell surface PAF receptor is in the cerebellum or the hippocampus.

46. The method of claim 41, wherein said blocking or inhibiting comprises administering a pharmaceutical composition or nutritional supplement to the lateral ventricle or the hippocampus.

47. A method of inhibiting angiogenesis in a subject, comprising blocking or inhibiting an intracellular platelet-activating factor (PAF) receptor, wherein said intracellular PAF receptor is in the spinal cord.

48. The method of claim 47, wherein said blocking or inhibiting comprises administering a pharmaceutical composition, said pharmacetical composition comprising BN 50730.

49. The method of claim 47, wherein said blocking or inhibiting comprises administering a pharmaceutical composition, said pharmacetical composition comprising a pharmaceutically acceptable carrier and a therapeutically effective amount of a benzodiazapine or a derivative thereof, either alone or in combination.

50. The method of claim 47, wherein said blocking or inhibiting comprises administering a pharmaceutical composition, said pharmacetical composition comprising a pharmaceutically acceptable carrier and a therapeutically effective amount of WEB-2086, WEB-2170, Y-24180, BN 50727, BN 50730, BN 50739, and E 6123, SM-12502, YM264, ABT-299, SR 27417, SRI 63-073, ONO-6240, RO-19 3704, UK-74,505, BB-882, or a derivative thereof, either alone or in combination.

51. A method of inhibiting neural damage in a subject, comprising blocking or inhibiting a cell surface platelet-activating factor (PAF) receptor, wherein said cell surface PAF receptor is in the brain.

52. The method of claim 51, wherein said blocking or inhibiting comprises administering a pharmaceutical composition, said pharmacetical composition comprising a pharmaceutically acceptable carrier and BN 52021.

53. The method of claim 51, wherein said blocking or inhibiting comprises administering a pharmaceutical composition, said pharmacetical composition comprising a pharmaceutically acceptable carrier and a therapeutically effective amount of CV-3988, CV-3938, CV-6209, TCV-309, E5880, SRI 63441, ginkgolide A, ginkgolide B, ginkgolide C, ginkgolide T, ginkgolide M, or a derivative thereof, either alone or in combination.

54. The method of claim 51, wherein said blocking or inhibiting comprises administering a nutritional supplement, said nutritional supplement comprising Gingko biloba, or derivatives or constitutents thereof.

55. The method of claim 51, wherein said cell surface PAF receptor is in the cerebellum or the hippocampus.

56. The method of claim 51, wherein said blocking or inhibiting comprises administering a pharmaceutical composition or nutritional supplement to the lateral ventricle or the hippocampus.

57. A method of inhibiting neural damage in a subject, comprising blocking or inhibiting an intracellular platelet-activating factor (PAF) receptor, wherein said intracellular PAF receptor is in the spinal cord.

58. The method of claim 57, wherein said blocking or inhibiting comprises administering a pharmaceutical composition, said pharmacetical composition comprising BN 50730.

59. The method of claim 57, wherein said blocking or inhibiting comprises administering a pharmaceutical composition, said pharmacetical composition comprising a pharmaceutically acceptable carrier and a therapeutically effective amount of a benzodiazapine or a derivative thereof, either alone or in combination.

60. The method of claim 57, wherein said blocking or inhibiting comprises administering a pharmaceutical composition, said pharmacetical composition comprising a pharmaceutically acceptable carrier and a therapeutically effective amount of WEB-2086, WEB-2170, Y-24180, BN 50727, BN 50730, BN 50739, and E 6123, SM-12502, YM264, ABT-299, SR 27417, SRI 63-073, ONO-6240, RO-19 3704, UK-74,505, BB-882, or a derivative thereof, either alone or in combination.

61. A method of treating pain, comprising blocking or inhibiting a platelet-activating factor (PAF) receptor.

62. The method of claim 61, wherein said blocking or inhibiting comprises administering a pharmaceutical composition, said pharmacetical composition comprising a pharmaceutically acceptable carrier and a therapeutically effective amount of a benzodiazapine, a tetrahydrofuran, or a derivative thereof, either alone or in combination.

63. The method of claim 61, wherein said blocking or inhibiting comprises administering a pharmaceutical composition, said pharmacetical composition comprising a pharmaceutically acceptable carrier and a therapeutically effective amount of BN 52021, BN 50730, WEB 286, CV 6209, CV 3988, trans-2,5-Bis(3,4,5-trimethoxypenyl)-1,3-dioxolane, 1-O-hexadecyl-2-O-acetyl-sn-glycero-3-phospho(N,N,N-trimethyl) hexanolamine, octylonium bromide, PCA-4248, tetrahydrocannabinol-7-oic acid, or a derivative thereof, either alone or in combination.

64. The method of claim 61, wherein said blocking or inhibiting comprises administering a pharmaceutical composition, said pharmacetical composition comprising a pharmaceutically acceptable carrier and a therapeutically effective amount of:
trans-2-(3-methoxy-5-methylsulfonyl-4-propoxyphenyl)-5(3,4,5-trimethoxyphenyl)tetrahydrofuran,
(2S, 3R, 4R)-tetrahydro-2-(3,4-dimethoxyphenyl)-4-(3,4-dimethoxybenzoyl)-3-(hydroxymethyl)furan,
(2S, 3R, 4R)-tetrahydro-2-(3,4,5-trimethoxyphenyl)-4-(3,4-dimethoxybenzoyl)-3-(hydroxymethyl)furan,
WEB-2086, WEB-2170, Y-24180, BN 50727, BN 50730, BN 50739, E 6123, CV-3988, CV-3938, CV-6209, TCV-309, E5880, SRI 63-441, SM-12502, YM264, ABT-299, SR 27417, UK-74,505, BB-882, WEB 2086, Y-24180, BN 50727, BN 50730, BN 50739, E 6123, or a derivative thereof.

65. The method of claim 61, wherein said blocking or inhibiting comprises administering a nutritional supplement, said nutritional supplement comprisingGinkgo biloba, Alphinia galanga, Boesenbergia pandurata, Curcuma aeruginosa, C. domestica, C. ochorrhiza, C. xanthorriza, Aingiber officinale, Z. zerumbet, Cinnamomum altissimum, C.aureofulvum, C. pubescens, Ardisia elliptica, Goniothalamus malayanus, Kopsia flavida, Momordica charantia, Piper aduncem, Drymis winteri, or derivatives or constitutents thereof, either alone or in combination.

66. The method of claim 61, wherein said blocking or inhibiting comprises administering a nutritional supplement, said nutritional supplement comprising Gingko biloba, or derivatives or constitutents thereof.

67. The method of claim 61, wherein said platelet-activating factor (PAF) receptor is selected from the group consisting of an intracellular PAF receptor and a cell surface PAF receptor.

68. The method of claim 67, further comprising blocking or inhibiting both an intracellular PAF receptor and said cell surface PAF receptor

69. The method of claim 67, further comprising blocking or inhibiting both a cell surface PAF receptor and said intracellular PAF receptor and said.

70. A method of treating inflammation, comprising blocking or inhibiting a platelet-activating factor (PAF) receptor.

71. The method of claim 70, wherein said blocking or inhibiting comprises administering a pharmaceutical composition, said pharmacetical composition comprising a pharmaceutically acceptable carrier and a therapeutically effective amount of a benzodiazapine, a tetrahydrofuran, or a derivative thereof, either alone or in combination.

72. The method of claim 70, wherein said blocking or inhibiting comprises administering a pharmaceutical composition, said pharmacetical composition comprising a pharmaceutically acceptable carrier and a therapeutically effective amount of BN 52021, BN 50730, WEB 286, CV 6209, CV 3988, trans-2,5-Bis(3,4,5-trimethoxypenyl)-1,3-dioxolane, 1-O-hexadecyl-2-O-acetyl-sn-glycero-3-phospho(N,N,N-trimethyl) hexanolamine, octylonium bromide, PCA-4248, tetrahydrocannabinol-7-oic acid, or a derivative thereof, either alone or in combination.

73. The method of claim 70, wherein said blocking or inhibiting comprises administering a pharmaceutical composition, said pharmacetical composition comprising a pharmaceutically acceptable carrier and a therapeutically effective amount of:
trans-2-(3-methoxy-5-methylsulfonyl-4-propoxyphenyl)-5(3,4,5-trimethoxyphenyl)tetrahydrofuran,
(2S, 3R, 4R)-tetrahydro-2-(3,4-dimethoxyphenyl)-4-(3,4-dimethoxybenzoyl)-3-(hydroxymethyl)furan,
(2S, 3R, 4R)-tetrahydro-2-(3,4,5-trimethoxyphenyl)-4-(3,4-dimethoxybenzoyl)-3-(hydroxymethyl)furan,
WEB-2086, WEB-2170, Y-24180, BN 50727, BN 50730, BN 50739, E 6123, CV-3988, CV-3938, CV-6209, TCV-309, E5880, SRI 63-441, SM-12502, YM264, ABT-299, SR 27417, UK-74,505, BB-882, WEB 2086, Y-24180, BN 50727, BN 50730, BN 50739, E 6123, or a derivative thereof, either alone or in combination.

74. The method of claim 70, wherein said blocking or inhibiting comprises administering a nutritional supplement, said nutritional supplement comprisingGinkgo biloba, Alphinia galanga, Boesenbergia pandurata, Curcuma aeruginosa, C. domestica, C. ochorrhiza, C. xanthorriza, Aingiber officinale, Z. zerumbet, Cinnamomum altissimum, C.aureofulvum, C. pubescens, Ardisia elliptica, Goniothalamus malayanus, Kopsia flavida, Momordica charantia, Piper aduncem, Drymis winteri, or derivatives or constitutents thereof, either alone or in combination.

75. The method of claim 70, wherein said blocking or inhibiting is achieved by administering a nutritional supplement, and wherein said nutritional is Gingko biloba, or derivatives or constitutents thereof.

76. The method of claim 70, wherein said PAF receptor is an intracellular PAF receptor.

77. The method of claim 70, wherein said inflammation comprises sepsis.

78. A method of inhibiting contraction of a uterus in a subject, comprising blocking or inhibiting a platelet-activating factor (PAF) receptor.

79. The method of claim 78, wherein said inhibiting contraction inhibits pain or cramps mediated by premenstrual syndrome, inhibits pain or cramps mediated by menses, inhibits spontaneous miscarriage, inhibits pain or cramps mediated by perimenopausal period, inhibits pain mediated by childbirth or that pain immediately following childbirth, or inhibits Braxton Hicks contractions.

80. The method of claim 78, wherein said blocking or inhibiting comprises administering a pharmaceutical composition, said pharmacetical composition comprising a pharmaceutically acceptable carrier and a therapeutically effective amount of a benzodiazapine, a tetrahydrofuran, or a derivative thereof, either alone or in combination.

81. The method of claim 78, wherein said blocking or inhibiting comprises administering a pharmaceutical composition, said pharmacetical composition comprising a pharmaceutically acceptable carrier and a therapeutically effective amount of BN 52021, BN 50730, WEB 286, CV 6209, CV 3988, trans-2,5-Bis(3,4,5-trimethoxypenyl)-1,3-dioxolane, 1-O-hexadecyl-2-O-acetyl-sn-glycero-3-phospho(N,N,N-triethyl) hexanolamine, octylonium bromide, PCA-4248, tetrahydrocannabinol-7-oic acid, or a derivative thereof, either alone or in combination.

82. The method of claim 78, wherein said blocking or inhibiting comprises administering a pharmaceutical composition, said pharmacetical composition comprising a pharmaceutically acceptable carrier and a therapeutically effective amount of:
trans-2-(3-methoxy-5-methylsulfonyl-4-propoxyphenyl)-5(3,4,5-trimethoxyphenyl)tetrahydrofuran,
(2S, 3R, 4R)-tetrahydro-2-(3,4-dimethoxyphenyl)-4-(3,4-dimethoxybenzoyl)-3-(hydroxymethyl)furan,
(2S, 3R, 4R)-tetrahydro-2-(3,4,5-trimethoxyphenyl)-4-(3,4-dimethoxybenzoyl)-3-(hydroxymethyl)furan,
WEB-2086, WEB-2170, Y-24180, BN 50727, BN 50730, BN 50739, E 6123, CV-3988, CV-3938, CV-6209, TCV-309, E5880, SRI 63-441, SM-12502, YM264, ABT-299, SR 27417, UK-74,505, BB-882, WEB 2086, Y-24180, BN 50727, BN 50730, BN 50739, E 6123, or a derivative thereof, either alone or in combination.

83. The method of claim 78, wherein said blocking or inhibiting is achieved by administering a nutritional supplement, and wherein said nutritional comprises Ginkgo biloba, Alphinia galanga, Boesenbergia pandurata, Curcuma aeruginosa, C. domestica, C. ochorriza, C. xanthorriza, Aingiber officinale, Z. zerumbet, Cinnamomum altissimum, C.aureofulvum, C. pubescens, Ardisia elliptica, Goniothalamus malayanus, Kopsia flavida, Momordica charantia, Piper aduncem, Drymis winteri, or derivatives or constituents thereof, either alone or in combination.

84. The method of claim 78, wherein said blocking or inhibiting is achieved by administering a nutritional supplement, and wherein said nutritional comprises Gingko biloba, or derivatives or constituents thereof.

85. A method of inhibiting proliferation of a tumor cell in a subject, comprising blocking or inhibiting a platelet-activating factor (PAF) receptor.

86. The method of claim 85, wherein said blocking or inhibiting comprises administering a pharmaceutical composition, said pharmacetical composition comprising a pharmaceutically acceptable carrier and a therapeutically effective amount of:
trans-2-(3-methoxy-5-methylsulfonyl-4-propoxyphenyl)-5(3,4,5-trimethoxyphenyl)tetrahydrofuran,
(2S, 3R, 4R)-tetrahydro-2-(3,4-diethoxyphenyl)-4-(3,4-dimethoxybenzoyl)-3-(hydroxymethyl)furan,
(2S, 3R, 4R)-tetrahydro-2-(3,4,5-trimethoxyphenyl)-4-(3,4-dimethoxybenzoyl)-3-(hydroxymethyl)furan,
WEB-2086, WEB-2170, Y-24180, BN 50727, BN 50730, BN 50739, E 6123, CV-3988, CV-3938, CV-6209, TCV-309, E5880, SRI 63-441, SM-12502, YM264, ABT-299, SR 27417, UK-74,505, BB-882, WEB 2086, Y-24180, BN 50727, BN 50730, BN 50739, E 6123, or a derivative thereof, either alone or in combination.

87. The method of claim 85, wherein said blocking or inhibiting is achieved by administering a nutritional supplement, and wherein said nutritional is Ginkgo biloba, Alphinia galanga, Boesenbergia pandurata, Curcuma aeruginosa, C. domestica, C. ochorrhiza, C. xanthorriza, Aingiber officinale, Z. zerumbet, Cinnamomum altissimum, C.aureofulvum, C. pubescens, Ardisia elliptica, Goniothalamus malayanus, Kopsia flavida, Momordica charantia, Piper aduncem, Drymis winteri, or derivatives or constituents thereof, either alone or in combination.

88. A method of inhibiting angiogenesis in a subject, comprising blocking or inhibiting a platelet-activating factor (PAF) receptor.

89. The method of claim 88, wherein said blocking or inhibiting comprises administering a pharmaceutical composition, said pharmacetical composition comprising a pharmaceutically acceptable carrier and a therapeutically effective amount of:
trans-2-(3-methoxy-5-methylsulfonyl-4-propoxyphenyl)-5(3,4,5-trimethoxyphenyl)tetrahydrofuran,
(2S, 3R, 4R)-tetrahydro-2-(3,4-dimethoxyphenyl)-4-(3,4-dimethoxybenzoyl)-3-(hydroxymethyl)furan,
(2S, 3R, 4R)-tetrahydro-2-(3,4,5-trimethoxyphenyl)-4-(3,4-dimethoxybenzoyl)-3-(hydroxymethyl)furan,
WEB-2086, WEB-2170, Y-24180, BN 50727, BN 50730, BN 50739, E 6123, CV-3988, CV-3938, CV-6209, TCV-309, E5880, SRI 63-441, SM-12502, YM264, ABT-299, SR 27417, UK-74,505, BB-882, WEB 2086, Y-24180, BN 50727, BN 50730, BN 50739, E 6123, or a derivative thereof, either alone or in combination.

90. The method of claim 88, wherein said blocking or inhibiting is achieved by administering a nutritional supplement, and wherein said nutritional comprises Ginkgo biloba, Alphinia galanga, Boesenbergia pandurata, Curcuma aeruginosa, C. domestica, C. ochorrhiza, C. xanthorriza, Aingiber officinale, Z. zerumbet, Cinnamomum altissimum, C.aureofulvum, C. pubescens, Ardisia elliptica, Goniothalamus malayanus, Kopsia flavida, Momordica charantia, Piper aduncem, Drymis winteri, or derivatives or constituents thereof, either alone or in combination.
